(19) United States
(12) Patent Application Publication
(10) Pub. No.: US 2008/0167330 A1
(43) Pub. Date: Jul. 10, 2008

(54) Carbamoylsulphoximides as protein kinase inhibitors

(76) Inventors: Ulrich Luecking, Berlin (DE); Gerhard Siemeister, Berlin (DE); Rolf Jautelat, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/892,967

(22) Filed: Aug. 28, 2007

Related U.S. Application Data
(60) Provisional application No. 60/841,567, filed on Sep. 1, 2006.

(30) Foreign Application Priority Data
Aug. 29, 2006 (DE) 10 2006 041 382.2

(51) Int. Cl. A61K 31/505; C07D 239/48; C07D 239/47; C07D 409/04; A61P 35/00; C07D 239/42; C07D 239/34; C07D 403/02; A61K 31/513; A61K 31/506

(52) U.S. Cl. 514/272; 544/323; 544/321; 514/275; 544/319; 544/326

(57) ABSTRACT

The invention relates to carbamoylsulphoximides as protein kinase inhibitors of the general formula 1.

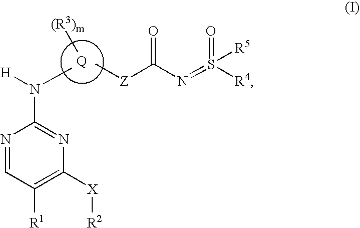

This application claims the benefit of the filing date of U.S. Provisional Application Ser. No. 60/841,567 filed Sep. 1, 2006.

The invention relates to sulphoximides as protein kinase inhibitors, in particular carbamoyl- and carbonyl sulphoximides.

Many biological processes such as, for example, DNA replication, energy metabolism, cell growth or cell differentiation in eukaryotic cells are regulated by reversible phosphorylation of proteins. The degree of phosphorylation of a protein has an influence inter alia on the function, localization or stability of proteins. The enzyme families of protein kinases and protein phosphatases are responsible respectively for the phosphorylation and dephosphorylation of proteins.

It is hoped, through inhibition of specific protein kinases or protein phosphatases, to be able to intervene in biological processes in such a way that causal or symptomatic treatment of diseases of the human or animal body is possible.

Protein kinases are of particular interest in this connection, inhibition thereof making the treatment of cancer possible.

The following protein kinase families come under consideration for example as targets for inhibitory molecules:
a) Cell cycle kinases, i.e. kinases whose activity control the progression of the cycle of cell division. Cell cycle kinases include substantially the cyclin-dependent kinases (cdk), the polo-like kinases (Plk), and the Aurora kinases.
b) Receptor tyrosine kinases which regulate angiogenesis (angiogenic receptor tyrosine kinases), such as, for example, the receptor tyrosine kinases which are involved in the vascular endothelial growth factor (VEGF)/VEGF receptor system, fibroblast growth factor (FGF)/FGF receptor system, in the Eph ligand/EphB4 system, and in the Tie ligand/Tie system,
c) Receptor tyrosine kinases whose activity contributes to the proliferation of cells (proliferative receptor tyrosine kinases), such as, for example, receptor tyrosine kinases which are involved in the platelet-derived growth factor (PDGF) ligand/PDGF receptor system, epithelial growth factor (EGF) ligand/EGF receptor system, c-Kit ligand/c-Kit receptor system and in the FMS-like tyrosine kinase 3 (Flt-3) ligand/Flt-3 system,
d) checkpoint kinases which monitor the ordered progression of cell division, such as, for example, ATM and ATR, Chk1 and Chk2, Mps1, Bub1 and BubR1,
e) kinases whose activity protects the cell from apoptosis (anti-apoptotic kinases, kinases in so-called survival pathways, anti-apoptotic kinases), such as, for example, Akt/PKB, PDK1, IkappaB kinase (IKK), Pim1, and integrin-linked kinase (ILK),
f) kinases which are necessary for the migration of tumour cells (migratory kinases), such as, for example, focal adhesion kinase (FAK) and Rho kinase (ROCK).

Inhibition of one or more of these protein kinases opens up the possibility of inhibiting tumour growth.

In this connection there is a need in particular for structures which, besides inhibiting cell cycle kinases, inhibit tumour growth through the inhibition of one or more further kinases (multi-target tumour growth inhibitors=MTGI). It is particularly preferred to inhibit in addition receptor tyrosine kinases which regulate angiogenesis.

The structures of the following patent applications form the structurally close prior art:

WO 2002/096888 discloses anilinopyrimidine derivatives as inhibitors of cyclin-dependent kinases. Carbamoylsulphoximide substituents are not disclosed for the aniline.

WO 2004/026881 discloses macrocyclic anilinopyrimidine derivatives as inhibitors of cyclin-dependent kinases. A possible carbamoylsulphoximide substituent for the aniline is not disclosed.

WO 2005/037800 discloses open anilinopyrimidine derivatives as inhibitors of cyclin-dependent kinases. Carbamoylsulphoximide substituents are not disclosed for the aniline.

It is common to all these structures of the prior art that they inhibit cell cycle kinases.

Starting from this prior art, it is the object of the present invention to provide a novel class of protein kinase inhibitors.

In particular, the object of the present invention is to provide inhibitors of protein kinases by which tumour growth can be inhibited.

There is a need in particular for a novel structural class which, besides cell cycle kinases, also inhibit receptor tyrosine kinases which inhibit angiogenesis.

The object of the present application is achieved by compounds of the general formula (I),

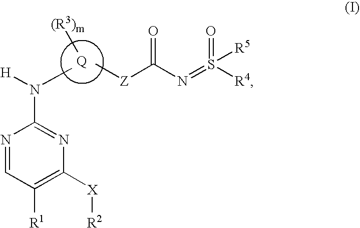

in which
R¹ is
   (i) hydrogen, halogen, cyano, nitro, —NR⁸R⁹, —NR⁷—C(O)—R¹², —NR⁷—C(O)—OR¹², —NR⁷—C(O)—NR⁸R⁹, —NR⁷—SO₂—R¹², —CF₃ or —OCF₃ or
   (ii) a C₁-C₆-alkyl, C₂-C₆-alkenyl, C₁-C₆-alkoxy or C₂-C₆-alkynyl radical which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, C₁-C₆-alkoxy, —CF₃ and/or —OCF₃, or
   (iii) a phenyl or monocyclic heteroaryl ring which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, —CF₃, C₁-C₆-alkoxy, —OCF₃ and/or C₁-C₆-alkyl,
R² is
   (i) hydrogen or
   (ii) a C₁-C₁₀-alkyl, C₂-C₁₀-alkenyl or C₂-C₁₀-alkynyl radical, a C₃-C₇-cycloalkyl, phenyl or naphthyl ring, a heterocyclyl ring having 3 to 8 ring atoms or a mono- or bicyclic heteroaryl ring,
   in each case optionally substituted one or more times, identically or differently, by
      a) halogen, hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, —C(O)R⁶, —O(CO)—R¹², —SO₂NR⁸R⁹, —SO₂—R¹², —S(O)(NR⁸)R¹², —(N)S(O)R¹³R¹⁴, —CF₃, —OCF₃, —N[(CO)—(C₁-C₆-alkyl)]₂ and/or
      b) C₁-C₆-alkoxy, C₁-C₆-alkyl, C₂-C₆-alkenyl, C₂-C₆-alkynyl, C₃-C₈-cycloalkyl, phenyl, naphthyl, heterocyclyl having 3 to 8 ring atoms and/or a monocyclic or bicyclic heteroaryl, in each case optionally themselves substituted one or more times, identically or differently, by halogen, hydroxy, a C₁-C₆-alkyl, C₁-C₆-alkoxy, —NR⁸R⁹, —C(O)OR¹⁶, —SO₂NR⁸R⁹, —CF₃ or —OCF₃,
R³ is
   (i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹, —NR⁷—SO₂—R¹² and/or
   (ii) a C₁-C₆-alkyl and/or C₁-C₆-alkoxy radical which is optionally substituted one or more times, identically or differently, by halogen, hydroxy, C₁-C₆-alkoxy, —CF₃, —OCF₃ or —NR⁸R⁹, and/or
   (iii) a C₃-C₇-cycloalkyl ring which is optionally substituted one or more times, identically or differently, by halogen, hydroxy, C₁-C₆-alkoxy, —CF₃, —OCF₃, —NR⁸R⁹ and/or C₁-C₆-alkyl,
m is 0-4,
z is the group —NH— or a direct linkage,
R⁴ and R⁵ are independently of one another a C₁-C₆-alkyl, C₂-C₆-alkenyl, C₂-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl,
   or
R⁴ and R⁵ together with the sulphur form a 3 to 7-membered ring which is optionally substituted one or more times, identically or differently, by hydroxy, C₁-C₆-alkyl, C₁-C₆-alkoxy, halogen or —NR⁸R⁹, and optionally comprises a double bond,
X is —O—, —S— or —NR¹⁵—,
   where
   R¹⁵ is
      (i) hydrogen or
      (ii) a C₁-C₆-alkyl radical, C₃-C₈-cycloalkyl or phenyl ring, a heterocyclyl ring having 3 to 8 ring atoms or a monocyclic heteroaryl ring, or
      (iii) —C(O)—(C₁-C₆)-alkyl, —C(O)-phenyl, or —C(O)-benzyl, and (ii) and (iii) are optionally substituted one or more times, identically or differently, by hydroxy, —NR¹⁰R¹¹, cyano, halogen, —CF₃, C₁-C₆-alkoxy and/or —OCF₃,
   or
   if X is —NR¹⁵—, alternatively
   —NR¹⁵— and R² together form a 3 to 8 membered ring which optionally comprises in addition to the nitrogen atom one or more further heteroatoms, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 to 3 double bonds, and/or is optionally interrupted by one or more —C(O)— groups,
Q is a phenyl, naphthyl or a monocyclic or bicyclic heteroaryl ring,
R⁶ is
   (i) hydrogen or hydroxy, or
   (ii) a C₁-C₆-alkyl, C₃-C₆-alkenyl, C₃-C₆-alkynyl or C₁-C₆-alkoxy radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
R⁷ is hydrogen or a C₁-C₆-alkyl radical,
R⁸ and R⁹ are independently of one another
   (i) hydrogen and/or
   (ii) a C₁-C₆-alkyl radical, C₂-C₆-alkenyl, C₃-C₈-cycloalkyl and/or phenyl ring, a heterocyclyl ring having 3 to 8 ring atoms and/or a monocyclic heteroaryl ring, optionally substituted one or more times, identically or differently, by hydroxy, —NR¹⁰R¹¹, cyano, halogen, —CF₃, C₁-C₆-alkoxy and/or —OCF₃,
   or
R⁸ and R⁹ together with the nitrogen atom form a 5- to 7-membered ring which optionally comprises in addition to the nitrogen atom 1 or 2 further heteroatoms, and which may be substituted one or more times, identically or differently, by hydroxy, —NR¹⁰R¹¹, cyano, halogen, —CF₃, C₁-C₆-alkoxy and/or —OCF₃,
R¹⁰ and R¹¹ are independently of one another hydrogen or a C₁-C₆-alkyl radical which is optionally substituted one or more times, identically or differently, by hydroxy, cyano, halogen, —CF₃, C₁-C₆-alkoxy and/or —OCF₃,
R¹², R¹³, R¹⁴ are independently of one another a C₁-C₆-alkyl, C₂-C₆-alkenyl and/or C₂-C₆-alkynyl radical, a C₃-C₇-cycloalkyl or phenyl ring, a heterocyclyl ring having 3 to 8 ring atoms or a monocyclic heteroaryl ring, optionally in each case themselves substituted one or more times, identically or differently, by hydroxy, nitro, —NR⁸R⁹, cyano, halogen, —CF₃, C₁-C₆-alkyl, C₁-C₆-alkoxy and/or —OCF₃,
R¹⁶ is
   (i) hydrogen or
   (ii) a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
      and the salts, diastereomers and enantiomers thereof.

Compounds in which Z is the —NH— group are referred to hereinafter as carbamoyl-sulphoximides and can be described by formula (Ia) in which all the radicals have the abovementioned meanings.

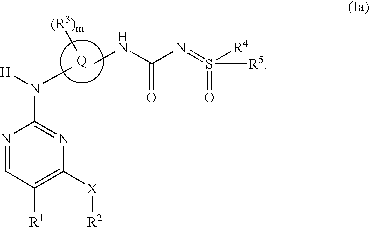

Compounds in which Z is a direct linkage are referred to hereinafter as carbonylsulphoximides and can be described by formula (Ib) in which all the radicals have the abovementioned meanings.

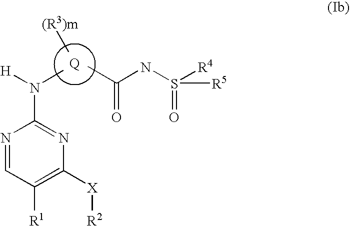

No prior art document proposes sulphoximide substituents on anilinopyrimidine derivatives which inhibit protein kinases. Nor are sulphoximide substituents disclosed for other structural classes which inhibit protein kinases.

The following definitions underlie the invention:

### Cₙ-Alkyl:

Monovalent, straight-chain or branched, saturated hydrocarbon radical having n carbon atoms.

A C₁-C₆ alkyl radical includes inter alia for example:

methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-1,2-dimethylbutyl-.

A methyl, ethyl, propyl or isopropyl radical is preferred.

### Cₙ-Alkenyl:

monovalent, straight-chain or branched hydrocarbon radical having n carbon atoms and at least one double bond.

A C₂-C₁₀ alkenyl radical includes inter alia for example:

vinyl-, allyl-, (E)-2-methylvinyl-, (Z)-2-methylvinyl-, homoallyl-, (E)-but-2-enyl-, (Z)-but-2-enyl-, (E)-but-1-enyl-, (Z)-but-1-enyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, isopropenyl-, 2-methylprop-2-enyl-, 1-methylprop-2-enyl-, 2-methylprop-1-enyl-, (E)-1-methylprop-1-enyl-, (Z)-1-methylprop-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, (E)-3,3-dimethylprop-1-enyl-, (Z)-3,3-dimethylprop-1-enyl-, 1-(1,1-dimethylethyl)ethenyl.

A vinyl or allyl radical is preferred.

### Cₙ-Alkynyl:

Monovalent, straight-chain or branched hydrocarbon radical having n carbon atoms and at least one triple bond.

A C₂-C₁₀ alkynyl radical includes inter alia for example:

ethynyl-, prop-1-ynyl-, prop-2-ynyl-, but-1-ynyl-, but-2-ynyl-, but-3-ynyl-, pent-1-ynyl-, pent-2-ynyl-, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- or a 3,3-dimethylbut-1-ynyl-.

An ethynyl, prop-1-ynyl or prop-2-ynyl radical is preferred.

### Cₙ-Cycloalkyl:

Monovalent, cyclic hydrocarbon ring having n carbon atoms.

C₃-C₇-Cycloalkyl ring includes:

cyclopropyl, cyclobutyl, cyclopentyl, cyclohexyl and cycloheptyl.

A cyclopropyl, cyclopentyl or a cyclohexyl ring is preferred.

### Cₙ-Alkoxy:

Straight-chain or branched Cₙ-alkyl ether residue of the formula —OR with R=alkyl.

### Cₙ-Aryl

Cₙ-Aryl is a monovalent, aromatic ring system without heteroatom having n hydrocarbon atoms.

C₆-Aryl is identical to phenyl. C₁₀-Aryl is identical to naphthyl.

Phenyl is preferred.

### Heteroatoms

Heteroatoms are to be understood to include oxygen, nitrogen or sulphur atoms.

### Heteroaryl

Heteroaryl is a monovalent, aromatic ring system having at least one heteroatom different from a carbon. Heteroatoms which may occur are nitrogen atoms, oxygen atoms and/or sulphur atoms. The valence bond may be on any aromatic carbon atom or on a nitrogen atom.

A monocyclic heteroaryl ring according to the present invention has 5 or 6 ring atoms.

Heteroaryl rings having 5 ring atoms include for example the rings:

thienyl, thiazolyl, furanyl, pyrrolyl, oxazolyl, imidazolyl, pyrazolyl, isoxazolyl, isothiazolyl, oxadiazolyl, triazolyl, tetrazolyl and thiadiazolyl.

Heteroaryl rings having 6 ring atoms include for example the rings:

pyridyl, pyridazinyl, pyrimidinyl, pyrazinyl and triazinyl.

A bicyclic heteroaryl ring according to the present invention has 9 or 10 ring atoms.

Heteroaryl rings having 9 ring atoms include for example the rings:

phthalidyl-, thiophthalidyl-, indolyl-, isoindolyl-, indazolyl-, benzothiazolyl-, indolonyl-, isoindolonyl-, benzofuranyl, benzothienyl, benzimidazolyl, benzoxazolyl, azocinyl, indolizinyl, purinyl.

Heteroaryl rings having 10 ring atoms include for example the rings:

isoquinolinyl-, quinolinyl-, benzoxazinonyl-, phthalazinonyl, quinolonyl-, isoquinolonyl-, quinazolinyl-, quinoxalinyl-, cinnolinyl-, phthalazinyl-, 1,7- or 1,8-naphthyridinyl-, quinolinyl-, isoquinolinyl-, quinazolinyl- or quinoxalinyl-

Monocyclic heteroaryl rings having 5 or 6 ring atoms are preferred.

### Heterocyclyl

Heterocyclyl in the context of the invention is a completely hydrogenated heteroaryl (completely hydrogenated heteroaryl=saturated heterocyclyl), i.e. a non-aromatic ring system having at least one heteroatom different from a carbon. Heteroatoms which may occur are nitrogen atoms, oxygen atoms and/or sulphur atoms. The valence bond may be on any carbon atom or on a nitrogen atom.

Heterocyclyl ring having 3 ring atoms includes for example:

aziridinyl.

Heterocyclyl ring having 4 ring atoms includes for example:

azetidinyl, oxetanyl.

Heterocyclyl rings having 5 ring atoms include for example the rings:

pyrrolidinyl, imidazolidinyl, pyrazolidinyl and tetrahydrofuranyl.

Heterocyclyl rings having 6 ring atoms include for example the rings:

piperidinyl, piperazinyl, morpholinyl, tetrahydropyranyl and thiomorpholinyl.

Heterocyclyl ring having 7 ring atoms includes for example:

azepanyl, oxepanyl, [1,3]-diazepanyl, [1,4]-diazepanyl.

Heterocyclyl ring having 8 ring atoms includes for example:

oxocanyl, azocanyl.

### Halogen

The term halogen includes fluorine, chlorine, bromine and iodine.

Bromine is preferred.

Preferred subgroups are compounds of the general formula (Ia) and (1b) in which
R¹ is halogen, —CF₃, —OCF₃, C₁-C₄-alkyl or nitro,
R² is a C₁-C₁₀-alkyl, C₂-C₁₀-alkenyl or C₂-C₁₀-alkynyl radical, a C₃-C₇-cycloalkyl, phenyl or a mono- or bicyclic heteroaryl ring or a heterocyclyl ring having 3 to 7 ring atoms,
   in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy
R³ is
   (i) hydroxy, halogen, cyano, nitro, —CF₃, —OCF₃, —NR⁸R⁹, —NR⁷—C(O)—R¹², —NR⁷—C(O)—OR¹², —NR⁷—C(O)—NR⁸R⁹, —NR⁷—SO₂—R¹², and/or
   (ii) a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,
   in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl,
   or
R⁴ and R⁵ together with the sulphur form a 3 to 7-membered ring which is optionally substituted one or more times, identically or differently, by hydroxy, C₁-C₆-alkyl, C₁-C₆-alkoxy or —NR⁸R⁹,
X is —O—, —S— or —NR¹⁵—,
   where
   R¹⁵ is
   (i) hydrogen or
   (ii) a C₁-C₆-alkyl radical, C₃-C₈-cycloalkyl or phenyl ring, a heterocyclyl ring having 3 to 8 ring atoms or a monocyclic heteroaryl ring, or
   (iii) —C(O)—(C₁-C₆)-alkyl, —C(O)-phenyl, or —C(O)-benzyl,
   and (ii) and (iii) are optionally substituted one or more times, identically or differently, by hydroxy, —NR¹⁰R¹¹, cyano, halogen, —CF₃, C₁-C₆-alkoxy and/or —OCF₃,
   or
   if X is —NR¹⁵—, alternatively
   —NR¹⁵— and R² together form a 3 to 8 membered ring which optionally comprises in addition to the nitrogen atom one or more further heteroatoms, is optionally substituted one or more times, identically or differently, by hydroxy, C₁-C₆-alkyl, C₁-C₆-alkoxy, —C(O)R¹², —SO₂R¹², halogen or the group —NR⁸R⁹, and/or is optionally interrupted by one or more —C(O)— groups,
Q is a phenyl or a monocyclic or bicyclic heteroaryl ring,
R⁶ is a C₂-C₅-alkyl, C₄-C₆-alkenyl, C₄-C₆-alkynyl or C₂-C₅-alkoxy radical, a C₄-C₆-cycloalkyl or phenyl ring, a heterocyclyl ring having 3 to 5 ring atoms or a monocyclic heteroaryl ring,
   in each case optionally themselves substituted one or more times, identically or differently, by hydroxy, —NR⁸R⁹, cyano, halogen, —CF₃, C₁-C₆-alkoxy and/or —OCF₃,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or phenyl ring, and/or a monocyclic heteroaryl ring,
   in each case optionally substituted one or more times, identically or differently, by hydroxy, —NR¹⁰R¹¹ or C₁-C₆-alkoxy,
   or
R⁸ and R⁹ together with the nitrogen atom form a 5- to 7-membered ring which optionally comprises in addition to the nitrogen atom 1 further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy,
R¹² is a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,
   in each case optionally themselves substituted one or more times, identically or differently, by hydroxy, halogen, nitro, —NR⁸R⁹, C₁-C₆-alkyl, and/or C₁-C₆-alkoxy,
R¹³ and R¹⁴ are independently of one another a C₁-C₆-alkyl radical, and
R¹⁶ is a C₁-C₆-alkyl radical, a C₃-C₇-cycloalkyl or phenyl ring, a heterocyclyl ring having 3 to 8 ring atoms or a monocyclic heteroaryl ring,
   and the salts, diastereomers and enantiomers thereof.

A particularly preferred subgroup are compounds of the general formula (I)

in which
R¹ is halogen, —CF₃ or a monocyclic heteroaryl ring which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, —CF₃, C₁-C₆-alkoxy, —OCF₃ and/or C₁-C₆-alkyl,
R² is a C₁-C₁₀-alkyl radical or bicyclic heteroaryl ring, in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy
R³ is halogen and/or a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl radical, in each case optionally itself substituted one or more times, identically or differently, by hydroxy, —NR⁸R⁹, C₁-C₆-alkoxy, and/or C₁-C₆-alkyl,
X is —O— or —NH—,
Q is a phenyl ring,
Z is the group —NH— or a direct linkage,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or a phenyl ring, and/or a monocyclic heteroaryl ring, in each case optionally substituted one or more times, identically or differently, by hydroxy, —NR¹⁰R¹¹ or C₁-C₆-alkoxy, or
R⁸ and R⁹ together with the nitrogen atom form a 5- to 7-membered ring which optionally comprises in addition to the nitrogen atom one further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy,
R¹² is a C₁-C₆-alkyl, C₂-C₆-alkenyl or C₂-C₆-alkynyl radical, a C₃-C₇-cyclo-alkyl or phenyl ring, a heterocyclyl ring having 3 to 8 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy,
   and the salts, diastereomers and enantiomers thereof.

A likewise particularly preferred subgroup are compounds of the general formula (Ia)

in which
R¹ is halogen or —CF₃
R² is a C₁-C₁₀-alkyl radical, in each case optionally substituted one or more times, identically or differently, by hydroxy, —NR⁸R⁹, —NR⁷—C(O)—R¹² and/or a C₁-C₄-alkyl radical which is optionally itself substituted one or more times by a hydroxy
R³ is halogen and/or a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl radical, in each case optionally itself substituted one or more times, identically or differently, by hydroxy, —NR⁸R⁹, C₁-C₆-alkoxy, and/or C₁-C₆-alkyl, or
X is —O— or —NH—,
Q is a phenyl ring,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or a phenyl ring, and/or a monocyclic heteroaryl ring, in each case optionally substituted one or more times, identically or differently, by hydroxy, —NR¹⁰R¹¹ or C₁-C₆-alkoxy, or
R⁸ and R⁹ together with the nitrogen atom form a 5- to 7-membered ring which optionally comprises in addition to the nitrogen atom one further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy.
R¹² is a C₁-C₆-alkyl, C₂-C₆-alkenyl or C₂-C₆-alkynyl radical, a C₃-C₇-cyclo-alkyl or phenyl ring, a heterocyclyl ring having 3 to 8 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy,
   and the salts, diastereomers and enantiomers thereof.

A likewise particularly preferred subgroup are compounds of the general formula (Ib) in which
R¹ is halogen or —CF₃
R² is a C₁-C₁₀-alkyl radical, in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy
m is 0,
R⁴ and R⁵ are independently of one another a C₁-C₆-alkyl radical, in each case optionally itself substituted one or more times, identically or differently, by hydroxy, —NR⁸R⁹, C₁-C₆-alkoxy, and/or C₁-C₆-alkyl, or
X is —O— or —NH—,
Q is a phenyl ring,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or a phenyl ring, and/or a monocyclic heteroaryl ring, in each case optionally substituted one or more times, identically or differently, by hydroxy, —NR¹⁰R¹¹ or C₁-C₆-alkoxy, or
R⁸ and R⁹ together with the nitrogen atom form a 5- to 7-membered ring which optionally comprises in addition to the nitrogen atom one further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy.
R¹² is a C₁-C₆-alkyl, C₂-C₆-alkenyl or C₂-C₆-alkynyl radical, a C₃-C₇-cyclo-alkyl or phenyl ring, a heterocyclyl ring having 3 to 8 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy,
   and the salts, diastereomers and enantiomers thereof.

A likewise particularly preferred subgroup are compounds of the general formula (1a)

in which
R¹ is halogen,
R² is a C₁-C₁₀-alkyl radical, optionally substituted one or more times, identically or differently, by hydroxy,
R³ is halogen and/or a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl radical, in each case optionally itself substituted one or more times, identically or differently, by hydroxy, —NR⁸R⁹, C₁-C₆-alkoxy, and/or C₁-C₆-alkyl, or
X is —O— or —NH—,
Q is a phenyl ring,
R⁸ and R⁹ are each independently of one another hydrogen and/or a C₁-C₄-alkyl radical, C₃-C₆-cycloalkyl and/or phenyl ring, and/or a monocyclic heteroaryl ring, in each case optionally substituted one or more times, identically or differently, by hydroxy or C₁-C₆-alkoxy, or
R⁸ and R⁹ together with the nitrogen atom form a 5- to 7-membered ring which optionally comprises in addition to the nitrogen atom one further heteroatom, and which may be substituted one or more times by hydroxy,
   and the salts, diastereomers and enantiomers thereof.

In the general formula (I), Q may be:

a phenyl, naphthyl or a monocyclic or bicyclic heteroaryl ring.
Q is preferably a phenyl or a monocyclic heteroaryl ring.
Q is more preferably a phenyl or a monocyclic heteroaryl ring having 6 ring atoms, in particular a pyridyl ring.
Q is particularly preferably a phenyl ring.

In the general formula (I), R¹ may be:
(i) hydrogen, halogen, cyano, nitro, —NR⁸R⁹, —NR⁷—C(O)—R¹², —NR⁷—C(O)—OR¹², —NR⁷—C(O)—NR⁸R⁹, —NR⁷—SO₂—R², —CF₃ or —OCF₃, or
(ii) a C₁-C₆-alkyl, C₂-C₆-alkenyl, C₁-C₆-alkoxy or C₂-C₆-alkynyl radical which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, C₁-C₆-alkoxy, —CF₃ and/or —OCF₃, or
(iii) a phenyl or monocyclic heteroaryl ring which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, —CF₃, C₁-C₆-alkoxy, —OCF₃ and/or C₁-C₆-alkyl.

R¹ is preferably:

halogen, —CF₃, —OCF₃, C₁-C₄-alkyl, nitro or a monocyclic heteroaryl ring which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, —CF₃, C₁-C₆-alkoxy, —OCF₃ and/or C₁-C₆-alkyl. R¹ is more preferably halogen, —CF₃, C₁-C₂-alkyl or a monocyclic heteroaryl ring which is optionally substituted one or more times, identically or differently, by hydroxy, cyano, halogen, —CF₃, C₁-C₆-alkoxy, —OCF₃ and/or C₁-C₆-alkyl.

R¹ is even more preferably halogen, —CF₃ or a monocyclic heteroaryl ring.

R¹ is particularly preferably —CF₃ or halogen, especially bromine.

In the general formula (I), R² may be:
(i) hydrogen or
(ii) a C₁-C₁₀-alkyl, C₂-C₁₀-alkenyl or C₂-C₁₀-alkynyl radical, a C₃-C₇-cycloalkyl, phenyl or naphthyl ring, a heterocyclyl ring having 3 to 8 ring atoms or a mono- or bicyclic heteroaryl ring,
   in each case optionally substituted one or more times, identically or differently, by
   a) halogen, hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, —C(O)R⁶, —O(CO)—R¹², —SO₂NR⁸R⁹, —SO₂—R¹², —S(O)(NR⁸)R¹², —(N)S(O)R¹³R¹⁴, —CF₃, —OCF₃, —N[(CO)—(C₁-C₆-alkyl)]₂ and/or
   b) C₁-C₆-alkoxy, C₁-C₆-alkyl, C₂-C₆-alkenyl, C₂-C₆-alkynyl, C₃-C₈-cycloalkyl, phenyl, naphthyl, heterocyclyl having 3 to 8 ring atoms and/or a monocyclic or bicyclic heteroaryl, in each case optionally themselves substituted one or more times, identically or differently, by halogen, hydroxy, a C₁-C₆-alkyl, C₁-C₆-alkoxy, —NR⁸R⁹, —C(O)OR¹⁶, —SO₂NR⁸R⁹, —CF₃ or —OCF₃.

R² is preferably:

a C₁-C₁₀-alkyl, C₂-C₁₀-alkenyl or C₂-C₁₀-alkynyl radical, a C₃-C₇-cycloalkyl, phenyl or a mono- or bicyclic heteroaryl ring, a heterocyclyl ring having 3 to 7 ring atoms, in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy.

R² is more preferably:

a C₂-C₆-alkyl, C₂-C₈-alkenyl or C₂-C₈-alkynyl radical, a C₃-C₆-cycloalkyl, phenyl ring, a bicyclic heteroaryl ring having 9 or 10 ring atoms, a heterocyclyl ring having 5 to 7 ring atoms, in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy.

R² is particularly preferably:

a C₂-C₆-alkyl radical or a bicyclic heteroaryl ring, having 9 or 10 ring atoms in each case optionally substituted one or more times, identically or differently, by hydroxy, NR⁸R⁹, —NR⁷—C(O)—R¹² and/or a C₁-C₄ alkyl radical which is optionally itself substituted one or more times by hydroxy.

R² is most preferably:

a C₂-C₆ alkyl radical, optionally substituted one or more times, identically or differently, by hydroxy.

In the general formula (I), X may be:

—O—, —S— or —NR¹⁵—,

where

R¹⁵ is
(i) hydrogen or
(ii) a C₁-C₆-alkyl radical, C₃-C₈-cycloalkyl or phenyl ring, a heterocyclyl ring having 3 to 8 ring atoms or a monocyclic heteroaryl ring, or
(iii) —C(O)—(C₁-C₆)-alkyl, —C(O)-phenyl, or —C(O)-benzyl,
   where (ii) and (iii) are optionally substituted one or more times, identically or differently, by hydroxy, —NR¹⁰R¹¹, cyano, halogen, —CF₃, C₁-C₆-alkoxy and/or —OCF₃,
      or
if X is —NR¹⁵—, alternatively —NR¹⁵— and R² together form a 3 to 8 membered ring which optionally comprises in addition to the nitrogen atom one or more further heteroatoms, is optionally substituted one or more times, identically or differently, by hydroxy, C₁-C₆-alkyl, C₁-C₆-alkoxy, —C(O)R¹²—SO₂R¹², halogen or the group —NR⁸R⁹, optionally comprises 1 to 3 double bonds, and/or is optionally interrupted by one or more —C(O)— groups.

X is preferably:

—O—, —S— or —NR¹⁵—, where

R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃,
or
if X is —NR¹⁵—, —NR¹⁵— and R² preferably alternatively together form a 3 to 6 membered ring which optionally comprises in addition to the nitrogen atom one further heteroatom, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 or 2 double bonds, and/or is interrupted by a —C(O)— group.

X is more preferably —NR¹⁵—,

where
R¹⁵ is hydrogen or a C₃-C₆-alkyl radical, C₃-C₇-cycloalkyl or a heterocyclyl ring having 3 to 6 ring atoms, in each case optionally substituted one or more times, identically or differently, by hydroxy, —NR¹⁰R¹¹, cyano, halogen, —CF₃, C₁-C₆-alkoxy and/or —OCF₃,
or
if X is —NR¹⁵—, —NR¹⁵— and R² more preferably alternatively together form a 5 or 6 membered ring which optionally comprises in addition to the nitrogen atom a further heteroatom, and which is optionally substituted one or more times, identically or differently, by hydroxy, C₁-C₆-alkyl, C₁-C₆-alkoxy, —C(O)R¹², —SO₂R¹², halogen or the group —NR⁸R⁹.

X is particularly preferably —O— or —NR¹⁵—, where R¹⁵ is hydrogen.

In the general formula (I), R³ can be:
(i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₆-alkyl and/or C₁-C₆-alkoxy radical which is optionally substituted one or more times, identically or differently, by halogen, hydroxy, C₁-C₆-alkoxy, —CF₃, —OCF₃ or —NR⁸R⁹, and/or
(iii) a C₃-C₇-cycloalkyl ring which is optionally substituted one or more times, identically or differently, by halogen, hydroxy, C₁-C₆-alkoxy, —CF₃, —OCF₃, —NR⁸R⁹ and/or C₁-C₆-alkyl.

R³ is preferably:
(i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₅-alkyl and/or C₁-C₅-alkoxy radical which is optionally substituted one or more times, identically or differently, by halogen, hydroxy, C₁-C₆-alkoxy, —CF₃, —OCF₃ or —NR⁸R⁹, and/or

R³ is more preferably
(i) hydroxy, halogen, cyano, nitro, —CF₃, —OCF₃, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹.

R³ is even more preferably:

(i) hydroxy, halogen, cyano, nitro, —CF₃, —OCF₃, —NR⁸R⁹ and/or
(ii) a C₁-C₃-alkyl and/or C₁-C₃-alkoxy radical.

R³ is particularly preferably:

halogen, is a C₁-C₃-alkyl and/or C₁-C₃-alkoxy radical and here is in particular fluorine, chlorine, methyl and/or methoxy.

In the general formula (I), m can be:

0-4, preferably 0-2, more preferably 0 or 1.

In the general formula (I), R⁴ and R⁵ can be independently of one another:

a C₁-C₆-alkyl, C₂-C₆-alkenyl, C₂-C₆-alkynyl radical, a C₃-C₇-cycloalkyl or phenyl ring, a heterocyclyl ring having 3 to 8 ring atoms or a monocyclic heteroaryl ring,
in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl,
or
R⁴ and R⁵ form together with the sulphur a 3 to 7-membered ring which is optionally substituted one or more times, identically or differently, by hydroxy, C₁-C₆-alkyl, C₁-C₆-alkoxy, halogen or —NR⁸R⁹, and optionally comprises a double bond.

R⁴ and R⁵ are preferably independently of one another:

a C₁-C₆-alkyl, C₂-C₆-alkenyl, C₂-C₆-alkynyl radical, a C₃-C₇-cycloalkyl or phenyl ring, a heterocyclyl ring having 3 to 8 ring atoms or a monocyclic heteroaryl ring,
in each case optionally themselves substituted one or more times, identically or differently, by hydroxy, —NR⁸R⁹, C₁-C₆-alkoxy and/or C₁-C₆-alkyl,
or
R⁴ and R⁵ form together with the sulphur a 3 to 7-membered ring which is optionally substituted one or more times, identically or differently, by hydroxy, C₁-C₆-alkyl, C₁-C₆-alkoxy and/or —NR⁸R⁹.

R⁴ and R⁵ are even more preferably independently of one another:

a C₁-C₅-alkyl, C₂-C₅-alkenyl, C₂-C₅-alkynyl radical, a C₃-C₆-cycloalkyl or phenyl ring, a heterocyclyl ring having 3 to 6 ring atoms or a monocyclic heteroaryl ring,
or
R⁴ and R⁵ form together with the sulphur a 3 to 7-membered ring.

R⁴ and R⁵ are particularly preferably independently of one another:

a C₁-C₄-alkyl, C₂-C₄-alkenyl, a C₃-C₇-cycloalkyl radical or a phenyl ring.

R⁴ and R⁵ are very particularly preferably independently of one another a C₁-C₆-alkyl radical.

R⁶ is preferably:
(i) hydrogen or
(ii) a C₁-C₄-alkyl, C₃-C₅-alkenyl, C₃-C₅-alkynyl or C₁-C₅-alkoxy radical, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R⁶ is more preferably:

a C₂-C₅-alkyl, C₄-C₆-alkenyl, C₄-C₆-alkynyl or C₂-C₅-alkoxy radical, a C₄-C₆-cycloalkyl or phenyl ring, a heterocyclyl ring having 3 to 5 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R⁶ is particularly preferably:

a C₁-C₆-alkyl, a C₁-C₆-alkoxy radical or a C₃-C₇-cycloalkyl ring, in each case optionally themselves substituted one or more times, identically or differently, by hydroxy, —NR⁸R⁹ and/or C₁-C₆-alkoxy.

R⁶ is very particularly preferably:

a C₁-C₆ alkyl or a C₁-C₆ alkoxy radical.

In the general formula (I), R⁷ may be hydrogen or a C₁-C₆-alkyl radical.

In the general formula (I), R⁸ and R⁹ may be independently of one another:
(i) hydrogen and/or
(ii) a C₁-C₆-alkyl radical, C₂-C₆-alkenyl, C₃-C₈-cycloalkyl and/or phenyl ring, a heterocyclyl ring having 3 to 8 ring atoms and/or a monocyclic heteroaryl ring, in each case optionally substituted one or more times, identically or differently, by hydroxy, —NR¹⁰R¹¹, cyano, halogen, —CF₃, C₁-C₆-alkoxy and/or —OCF₃,
   or
   R⁸ and R⁹ form together with the nitrogen atom a 5- to 7-membered ring which optionally comprises in addition to the nitrogen atom 1 or 2 further heteroatoms, and which may be substituted one or more times, identically or differently, by hydroxy, —NR¹⁰R¹¹, cyano, halogen, —CF₃, C₁-C₆-alkoxy and/or —OCF₃.

R⁸ and R⁹ are preferably:
(i) hydrogen and/or
(ii) a C₁-C₅-alkyl, C₂-C₅-alkenyl radical, a C₃-C₇-cycloalkyl and/or phenyl ring and/or a monocyclic heteroaryl ring, in each case optionally substituted one or more times, identically or differently, by hydroxy, —NR¹⁰R¹¹ and/or C₁-C₆-alkoxy,
   or
   R⁸ and R⁹ form together with the nitrogen atom a 5- to 7-membered ring which optionally comprises in addition to the nitrogen atom 1 further heteroatom and which may be substituted one or more times, identically or differently, by hydroxy, —NR¹⁰R¹¹ and/or C₁-C₆-alkoxy.

R⁸ and R⁹ are more preferably:
(i) hydrogen and/or
(ii) a C₁-C₄-alkyl radical, C₃-C₆-cycloalkyl and/or phenyl ring, and/or a monocyclic heteroaryl ring, in each case optionally substituted one or more times, identically or differently, by hydroxy, —NR¹⁰R¹¹ or C₁-C₆-alkoxy,
   or
   R⁸ and R⁹ form together with the nitrogen atom a 5- to 7-membered ring which optionally comprises in addition to the nitrogen atom 1 further heteroatom, and which may be substituted one or more times by hydroxy.

R⁸ and R⁹ are particularly preferably:
(i) hydrogen and/or
(ii) a C₁-C₆-alkyl radical, a C₃-C₆-cycloalkyl and/or phenyl ring and/or a monocyclic heteroaryl ring,
   or
   R⁸ and R⁹ form together with the nitrogen atom a 5- or 6-membered ring which optionally comprises in addition to the nitrogen atom 1 further heteroatom.

In the general formula (I), R¹⁰ and R¹¹ may be independently of one another hydrogen or a C₁-C₆-alkyl radical which is optionally substituted one or more times, identically or differently, by hydroxy, cyano, halogen, —CF₃, C₁-C₆-alkoxy and/or —OCF₃.

R¹⁰ and R¹¹ may preferably independently of one another be hydrogen or a C₁-C₆-alkyl radical which is optionally substituted one or more times, identically or differently, by hydroxy, halogen or C₁-C₆-alkoxy.

R¹⁰ and R¹¹ may more preferably be independently of one another hydrogen or a C₁-C₆-alkyl radical which is optionally substituted one or more times, identically or differently, by hydroxy.

R¹⁰ and R¹¹ may particularly preferably be independently of one another hydrogen or a methyl group.

In the general formula (I), R¹², R¹³, R¹⁴ may be independently of one another a C₁-C₆-alkyl, C₂-C₆-alkenyl and/or C₂-C₆-alkynyl radical, a C₃-C₇-cycloalkyl and/or phenyl ring, a heterocyclyl ring having 3 to 8 ring atoms and/or a monocyclic heteroaryl ring,

in each case optionally themselves substituted one or more times, identically or differently, by hydroxy, nitro, —NR⁸R⁹, cyano, halogen, —CF₃, C₁-C₆-alkyl, C₁-C₆-alkoxy and/or —OCF₃.

R¹² is preferably a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,

in each case optionally themselves substituted one or more times, identically or differently, by hydroxy, halogen, nitro, —NR⁸R⁹, C₁-C₆-alkyl and/or C₁-C₆-alkoxy.

R¹² is more preferably a C₁-C₅-alkyl, C₂-C₅-alkenyl, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy.

R¹² is particularly preferably a C₁-C₆-alkyl radical, a phenyl or monocyclic heteroaryl ring,

in each case optionally themselves substituted one or more times, identically or differently, by hydroxy, halogen or C₁-C₆-alkyl.

R¹³ and R¹⁴ are preferably independently of one another a C₁-C₆-alkyl, C₂-C₆-alkenyl and/or C₂-C₆-alkynyl radical, a C₃-C₇-cycloalkyl and/or phenyl ring, a heterocyclyl ring having 3 to 8 ring atoms and/or a monocyclic heteroaryl ring, in each case optionally themselves substituted one or more times, identically or differently, by hydroxy, —NR⁸R⁹ and/or C₁-C₆-alkoxy.

R¹³ and R¹⁴ are more preferably independently of one another a C₁-C₅-alkyl, C₂-C₅-alkenyl and/or C₂-C₅-alkynyl radical, a C₃-C₆-cycloalkyl and/or phenyl ring, a heterocyclyl ring having 3 to 6 ring atoms and/or a monocyclic heteroaryl ring.

R¹³ and R¹⁴ are particularly preferably independently of one another a C₁-C₆-alkyl radical.

R¹³ and R¹⁴ are very particularly preferably a methyl radical.

In the general formula (I), R¹⁶ may be:
(i) hydrogen or
(ii) a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R¹⁶ may preferably be:

a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R¹⁶ can more preferably be:

a C₁-C₆-alkyl radical, a C₃-C₇-cycloalkyl or phenyl ring, a heterocyclyl ring having 3 to 8 ring atoms or a monocyclic heteroaryl ring.

R¹⁶ may particularly preferably be a C₁-C₆-alkyl radical.

Likewise to be regarded as encompassed by the present invention are all compounds which result from every possible combination of the above-mentioned possible, preferred and particularly preferred meanings of the substituents.

Special embodiments of the invention moreover consist of compounds which result from combination of the meanings disclosed directly in the examples for the substituents.

The compounds of the formula (I) according to the invention can be prepared by reacting 2-chloropyrimidines of the formula (II) with nucleophiles of the formula (III) to give compounds of the formula (I)

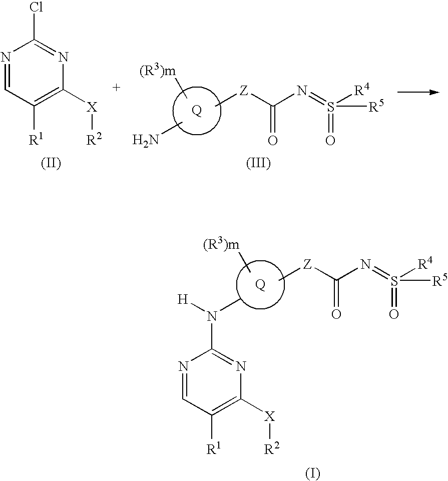

where Q, R¹, R², R³, R⁴, R⁵, X, Z and m have the meanings indicated in the general formula (I) according to claims 1 to 18.

The present invention likewise relates to intermediates of the formula (II):

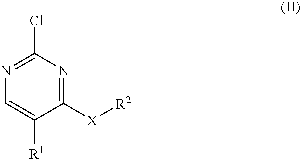

where R¹, R² and X have the meanings indicated in the general formula (I) according to claims 1 to 18.

The intermediates of the formula (II) can be prepared by reacting 2,4-dichloro-pyrimidines of the formula (V) with nucleophiles of the formula (IV)

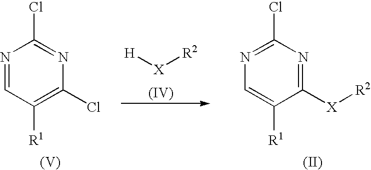

where R¹, R² and X have the meanings indicated in the general formula (I) according to claims 1 to 18.

The present invention likewise relates to intermediates compounds of the formula (III), in particular of the formula (IIIa) and (IIIb):

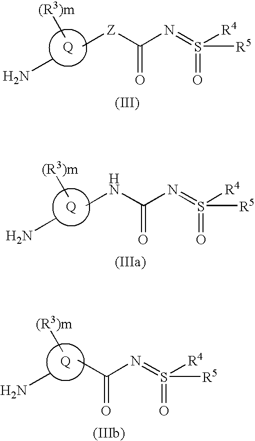

where Q, Z, R³, R⁴ and R⁵ have the meanings indicated in the general formula (I) according to claims 1 to 18.

The intermediates of the formula (IIIa) can be prepared by a process which includes the following steps:
a) reaction of an isocyanate of the formula (VII) with a sulphoximine of the formula (VIII) to give an intermediate of the formula (VI)

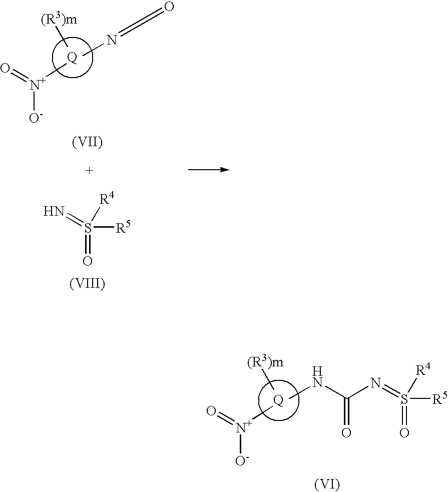

b) a reduction of the nitro group to result in the intermediates of the formula (IIIa)

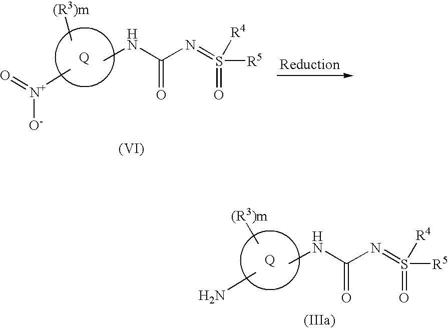

where Q, R³, R⁴ and R⁵ have the meanings indicated in the general formula (I) according to claims 1 to 18.

The intermediates of the formula (IIIb) can be prepared by a process which includes the following steps:
a) reaction of an acid chloride of the formula (IX) with a sulphoximine of the formula (VIII) to give intermediates of the formula (X)

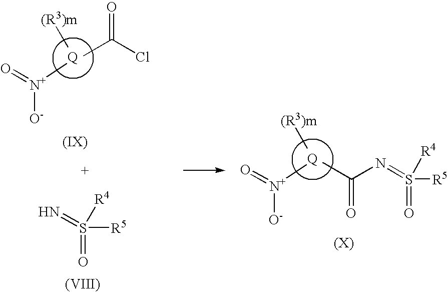

b) a reduction of the nitro group to result in the intermediates of the formula (IIIb)

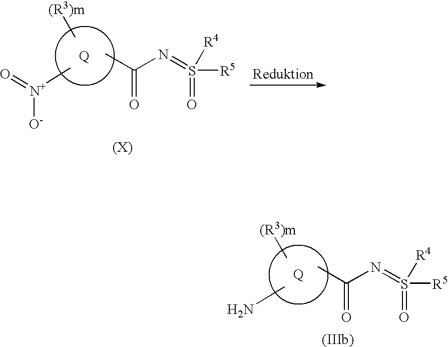

where Q, R³, R⁴ and R⁵ have the meanings indicated in the general formula (I) according to claims 1 to 18.

The following grouping of protein kinases underlies the application:

A. cell cycle kinases: a) CDKs, b) Plk, c) Aurora
B. angiogenic receptor tyrosine kinases: a) VEGF-R, b) Tie, c) FGF-R, d) EphB4
C. proliferative receptor tyrosine kinases: a) PDGF-R, Flt-3, c-Kit
D. checkpoint kinases: a) AMT/ATR, b) Chk 1/2, c) TTK/hMps1, BubR1, Bub1
E. anti-apoptotic kinases a) AKT/PKB b) IKK c) PIM1, d) ILK
F. migratory kinases a) FAK, b) ROCK

### A. Cell Cycle Kinases a) CDKs, b) Plk, c) Aurora

The eukaryotic cycle of cell division ensures duplication of the genome and its distribution to the daughter cells by passing through a coordinated and regulated sequence of events. The cell cycle is divided into four consecutive phases: the G1 phase represents the time before DNA replication in which the cell grows and is sensitive to external stimuli. In the S phase, the cell replicates its DNA, and in the G2 phase it prepares itself for entry into mitosis. In mitosis (M phase), the replicated DNA is separated and cell division is completed.

The cyclin-dependent kinases (CDKs), a family of serine/threonine kinases whose members require the binding of a cyclin (Cyc) as regulatory subunit for their activation, drive the cell through the cell cycle. Different CDK/Cyc pairs are active in the different phases of the cell cycle. CDK/Cyc pairs which are important for the basic function of the cell cycle are, for example, CDK4(6)/CycD, CDK2/CycE, CDK2/CycA, CDK1/CycA and CDK1/CycB.

Entry into the cell cycle and passing through the restriction point, which marks the independence of a cell from further growth signals for completion of the initiated cell division, are controlled by the activity of the CDK4(6)/CycD and CDK2/CycE complexes. The essential substrate of these CDK complexes is the retinoblastoma protein (Rb), the product of the retinoblastoma tumour suppressor gene. Rb is a transcriptional corepressor protein. Besides other mechanisms which are still substantially not understood, Rb binds and inactivates transcription factors of the E2F type, and forms transcriptional repressor complexes with histone deacetylases (HDAC) (Zhang H. S. et al. (2000). Exit from G1 and S phase of the cell cycle is regulated by repressor complexes containing HDAC-Rb-hSWI/SNF and Rb-hSWI/SNF. Cell 101, 79-89). Phosphorylation of Rb by CDKs releases bound E2F transcription factors which lead to transcriptional activation of genes whose products are required for DNA synthesis and progression through the S phase. An additional effect of Rb phosphorylation is to break up Rb-HDAC complexes, thus activating further genes. Phosphorylation of Rb by CDKs is to be equated with going beyond the restriction point. The activity of CDK2/CycE and CDK2/CycA complexes is necessary for progression through the S phase and completion thereof. After replication of the DNA is complete, the CDK1 in the complex with CycA or CycB controls the passing through of the G2 phase and the entry of the cell into mitosis (FIG. 1). In the transition from the G2 phase into mitosis, the polo-like kinase Plk1 contributes to activating CDK1. While mitosis is in progress, Plk1 is further involved in the maturation of the centrosomes, the construction of the spindle apparatus, the separation of the chromosomes and the separation of the daughter cells.

The family of Aurora kinases consists in the human body of three members:

Aurora-A, Aurora-B and Aurora-C. The Aurora kinases regulate important processes during cell division (mitosis).

Aurora-A is localized on the centrosomes and the spindle microtubules, where it phosphorylates various substrate proteins, inter alia kinesin Eg5, TACC, PP1. The exact mechanisms of the generation of the spindle apparatus and the role of Aurora-A therein are, however, still substantially unclear.

Aurora-B is part of a multiprotein complex which is localized on the centrosome structure of the chromosomes and, besides Aurora-B, comprises inter alia INCENP, survivin and borealin/dasra B (summarizing overview in: Vagnarelli & Earnshaw, Chromosomal passengers: the four-dimensional regulation of mitotic events. Chromosoma. 2004 November; 113(5):211-22. Epub 2004 Sep. 4). The kinase activity of Aurora-B ensures that all the connections to the microtubulin spindle apparatus are correct before division of the pairs of chromosomes (so-called spindle checkpoint). Substrates of Aurora-B are in this case inter alia histone H3 and MCAK. After separation of the chromosomes, Aurora-B alters its localization and can be found during the last phase of mitosis (cytokinesis) on the still remaining connecting bridge between the two daughter cells. Aurora-B regulates the severance of the daughter cells through phosphorylation of its substrates MgcRacGAP, vimentin, desmin, the light regulatory chain of myosin, and others.

Aurora-C is very similar in its amino acid sequence, localization, substrate specificity and function to Aurora-B (Li X et al. Direct association with inner centromere protein (INCENP) activates the novel chromosomal passenger protein, Aurora-C. J Biol. Chem. 2004 Nov. 5; 279(45):47201-11. Epub 2004 Aug. 16; Chen et al. Overexpression of an Aurora-C kinase-deficient mutant disrupts the Aurora-B/INCENP complex and induces polyploidy. J Biomed Sci. 2005; 12(2):297-310; Yan X et al. Aurora-C is directly associated with Survivin and required for cytokinesis. Genes to ells 2005 10, 617-626). The chief difference between Aurora-B and Aurora-C is the strong overexpression of Aurora-C in the testis (Tseng T C et al. Protein kinase profile of sperm and eggs: cloning and characterization of two novel testis-specific protein kinases (AIE1, AIE2) related to yeast and fly chromosome segregation regulators. DNA Cell Biol. 1998 October; 17(10):823-33).

The essential function of the Aurora kinases in mitosis makes them target proteins of interest for the development of small inhibitory molecules for the treatment of cancer or other disorders which are caused by disturbances of cell proliferation. Convincing experimental data indicate that inhibition of the Aurora kinases in vitro and in vivo prevents the advance of cellular proliferation and induces programmed cell death (apoptosis). It has been possible to show this by means of (1) siRNA technology (Du & Hannon. Suppression of p160ROCK bypasses cell cycle arrest after Aurora-A/STK15 depletion. Proc Natl Acad Sci USA. 2004 Jun. 15; 101(24):8975-80. Epub 2004 Jun. 3; Sasai K et al. Aurora-C kinase is a novel chromosomal passenger protein that can complement Aurora-B kinase function in mitotic cells. Cell Motil Cytoskeleton. 2004 December; 59(4):249-63) or (2) overexpression of a dominant-negative Aurora kinase (Honda et al. Exploring the functional interactions between Aurora B, INCENP, and survivin in mitosis. Mol Biol Cell. 2003 August; 14(8):3325-41. Epub 2003 May 29), and (3) with small chemical molecules which specifically inhibit Aurora kinases (Hauf S et al. The small molecule Hesperadin reveals a role for Aurora B in correcting kinetochore-microtubule attachment and in maintaining the spindle assembly checkpoint. J Cell Biol. 2003 Apr. 28; 161(2):281-94. Epub 2003 Apr. 21; Ditchfield C et al. Aurora B couples chromosome alignment with anaphase by targeting BubR1, Mad2, and Cenp-E to kinetochores. J Cell Biol. 2003 Apr. 28; 161(2):267-80).

Inactivation of Aurora kinases leads to (1) faulty or no development of the mitotic spindle apparatus (predominantly with Aurora-A inhibition) and/or (2) faulty or no separation of the sister chromatids through blocking of the spindle checkpoint (predominantly with Aurora-B/-C inhibition) and/or (3) incomplete separation of daughter cells (predominantly with Aurora-B/-C inhibition). These consequences (1-3) of the inactivation of Aurora kinases singly or as combinations lead eventually to aneuploidy and/or polyploidy and ultimately, immediately or after repeated mitoses, to a non-viable state or to programmed cell death of the proliferating cells (mitotic catastrophe).

Specific kinase inhibitors are able to influence the cell cycle at various stages. Thus, for example, blockade of the cell cycle in the G1 phase or in the transition from the G1 phase to the S phase is to be expected with a CDK4 or a CDK2 inhibitor.

### B. Angiogenic Receptor Tyrosine Kinases

Receptor tyrosine kinases and their ligands are crucial participants in a large number of cellular processes involved in the regulation of the growth and differentiation of cells. Of particular interest here are the vascular endothelial growth factor (VEGF)/VEGF receptor system, the fibroblast growth factor (FGF)/FGF receptor system, the Eph ligand/Eph receptor system, and the Tie ligand/Tie receptor system. In pathological situations associated with an increased formation of new blood vessels (neovascularization) such as, for example, neoplastic diseases, an increased expression of angiogenic growth factors and their receptors has been found. Inhibitors of the VEGF/VEGF receptor system, FGF/FGF receptor system (Rousseau et al., The tyrp1-Tag/tyrp1-FGFR1-DN bigenic mouse: a model for selective inhibition of tumor development, angiogenesis, and invasion into the neural tissue by blockade of fibroblast growth factor receptor activity. Cancer Res. 64, 2490, 2004), of the EphB4 system (Kertesz et al., The soluble extracellular domain of EphB4 (sEphB4) antagonizes EphB4-EphrinB2 interaction, modulates angiogenesis and inhibits tumor growth. Blood. 2005 Dec. 1; [Epub ahead of print]), and of the Tie ligand/Tie system (Siemeister et al., Two independent mechanisms essential for tumor angiogenesis: inhibition of human melanoma xenograft growth by interfering with either the vascular endothelial growth factor receptor pathway or the Tie-2 pathway. Cancer Res. 59, 3185, 1999) are able to inhibit the development of a vascular system in tumours, thus cut the tumour off from the oxygen and nutrient supply, and therefore inhibit tumour growth.

### C. Proliferative Receptor Tyrosine Kinases

Receptor tyrosine kinases and their ligands are crucial participants in the proliferation of cells. Of particular interest here are the platelet-derived growth factor (PDGF) ligand/PDGF receptor system, c-kit ligand/c-kit receptor system and the FMS-like tyrosine kinase 3 (Flt-3) ligand/Flt-3 system. In pathological situations associated with an increased growth of cells such as, for example, neoplastic diseases, an increased expression of proliferative growth factors and their receptors or kinase-activating mutations has been found. Inhibition of the enzymic activity of these receptor tyrosine kinases leads to a reduction of tumour growth. It has been possible to show this for example by studies with the small chemical molecule STI571/Glivec which inhibits inter alia PDGF-R and c-kit (summarizing overviews in: Oestmann A., PDGF receptors—mediators of autocrine tumor growth and regulators of tumor vasculature and stroma, Cytokine Growth Factor Rev. 2004 August; 15(4):275-86; Roskoski R., Signaling by Kit protein-tyrosine kinase—the stem cell factor receptor. Biochem Biophys Res Commun. 2005 Nov. 11; 337(1):1-13; Markovic A. et al., FLT-3: a new focus in the understanding of acute leukemia. Int J Biochem Cell Biol. 2005 June; 37(6):1168-72. Epub 2005 Jan. 26).

### E. Checkpoint Kinases

Checkpoint kinases mean in the context of the present application cell cycle kinases which monitor the ordered progression of cell division, such as, for example, ATM and ATR, Chk1 and Chk2, Mps1, Bub1 and BubR1. Of particular importance are the DNA damage checkpoint in the G2 phase and the spindle checkpoint during mitosis.

The ATM, ATR, Chk1 and Chk2 kinases are activated by DNA damage to a cell and leads to arrest of the cell cycle in the G2 phase through inactivation of CDK1. (Chen & Sanchez, Chk1 in the DNA damage response: conserved roles from yeasts to mammals. DNA Repair 3, 1025, 2004). Inactivation of Chk1 causes loss of the G2 arrest induced by DNA damage, to progression of the cell cycle in the presence of damaged DNA, and finally leads to cell death (Takai et al. Aberrant cell cycle checkpoint function and early embryonic death in Chk1(−/−) mice. Genes Dev. 2000 Jun. 15; 14(12):1439-47; Koniaras et al. Inhibition of Chk1-dependent G2 DNA damage checkpoint radiosensitizes p53 mutant human cells. Oncogene. 2001 Nov. 8; 20(51):7453-63; Liu et al. Chk1 is an essential kinase that is regulated by Atr and required for the G(2)/M DNA damage checkpoint. Genes Dev. 2000 Jun. 15; 14(12):1448-59). Inactivation of Chk1, Chk2 or Chk1 and Chk2 prevents the G2 arrest caused by DNA damage and makes proliferating cancer cells more sensitive to DNA-damaging therapies such as, for example, chemotherapy or radiotherapy. Chemotherapies leading to DNA damage are, for example, substances inducing DNA strand breaks, DNA-alkylating substances, topoisomerase inhibitors, Aurora kinase inhibitors, substances which influence the construction of the mitotic spindles, hypoxic stress owing to a limited oxygen supply to a tumour (e.g. induced by anti-angiogenic medicaments such as VEGF kinase inhibitors).

A second essential checkpoint within the cell cycle controls the correct construction and attachment of the spindle apparatus to the chromosomes during mitosis. The kinases TTK/hMps1, Bub1, and BubR1 are involved in this so-called spindle checkpoint (summarizing overview in: Kops et al. On the road to cancer: aneuploidy and the mitotic checkpoint. Nat Rev Cancer. 2005 October; 5(10):773-85). These are localized on kinetochores of condensed chromosomes which are not yet attached to the spindle apparatus and inhibit the so-called anaphase-promoting complex/cyclosome (APC/C). Only after complete and correct attachment of the spindle apparatus to the kinetochores are the spindle checkpoint kinases Mps-1, Bub1, and BubR1 inactivated, thus activating APC/C and resulting in separation of the paired chromosomes. Inhibition of the spindle checkpoint kinases leads to separation of the paired chromosomes before all the kinetochores are attached to the spindle apparatus, and consequently to faulty chromosome distributions which are not tolerated by cells and finally lead to cell cycle arrest or cell death.

### F. Anti-Apoptotic Kinases

Various mechanisms protect a cell from cell death during non-optimal living conditions. In tumour cells, these mechanisms lead to a survival advantage of the cells in the growing mass of the tumour, which is characterized by deficiency of oxygen, glucose and further nutrients, make it possible for tumour cells to survive without attachment to the extracellular matrix, possibly leading to metastasis, or lead to resistances to therapeutic agents. Essential anti-apoptotic signalling pathways include the PDK1-AKT/PKB signalling pathway (Altomare & Testa. Perturbations of the AKT signaling pathway in human cancer. Oncogene. 24, 7455, 2005), the NFkappaB signalling pathway (Viatour et al. Phosphorylation of NFkB and IkB proteins: implications in cancer and inflammation), the Pim1 signalling pathway (Hammerman et al. Pim and Akt oncogenes are independent regulators of hematopoietic cell growth and survival. Blood. 2005 105, 4477, 2005) and the integrin-linked kinase (ILK) signalling pathway (Persad & Dedhar. The role of integrin-linked kinase (ILK) in cancer progression. Cancer Met. Rev. 22, 375, 2003). Inhibition of the anti-apoptotic kinases such as, for example, AKT/PBK, PDK1, IkappaB kinase (IKK), Pim1, or ILK sensitizes the tumour cells to the effect of therapeutic agents or to unfavourable living conditions in the tumour environment. After inhibition of the anti-apoptotic kinases, tumour cells will react more sensitively to disturbances of mitosis caused by Aurora inhibition and undergo cell death in increased numbers.

### G. Migratory Kinases

A precondition for invasive, tissue-infiltrating tumour growth and metastasis is that the tumour cells are able to leave the tissue structure through migration. Various cellular mechanisms are involved in regulating cell migration: integrin-mediated adhesion to proteins of the extracellular matrix regulates via the activity of focal adhesion kinase (FAK); control of the assembling of contractile actin filaments via the RhoA/Rho kinase (ROCK) signalling pathway (summarizing overview in M. C. Frame, Newest findings on the oldest oncogene; how activated src does it. J. Cell Sci. 117, 989, 2004).

The compounds according to the invention are effective for example
against cancer such as solid tumours, tumour growth or metastasis growth, especially:
ataxia-telangiectasia, basal cell carcinoma, bladder carcinoma, brain tumour, breast cancer, cervical carcinoma, tumours of the central nervous system, colorectal carcinoma, endometrial carcinoma, stomach carcinoma, gastrointestinal carcinoma, head and neck tumours, acute lymphocytic leukaemia, acute myelogenous leukaemia, chronic lymphocytic leukaemia, chronic myelogenous leukaemia, hairy cell leukaemia, liver carcinoma, lung tumour, non-small-cell lung carcinoma, small-cell lung carcinoma, B-cell lymphoma, Hodgkin's lymphoma, non-Hodgkin's lymphoma, T-cell lymphoma, melanoma, mesothelioma, myeloma, myoma, tumours of the oesophagus, oral tumours, ovarian carcinoma, pancreatic tumours, prostate tumours, renal carcinoma, sarcoma, Kaposi's sarcoma, leiomyosarcoma, skin cancer, squamous cell carcinoma, testicular cancer, thyroid cancer, connective tissue tumour of the gastrointestinal tissue, connective tissue sarcoma of the skin, hypereosinophilic syndrome, mast cell cancer,
for cardiovascular disorders such as stenoses, arterioscleroses and restenoses, stent-induced restenosis,
for angiofibroma, Crohn's disease, endometriosis, haemangioma.

Formulation of the compounds according to the invention to give pharmaceutical products takes place in a manner known per se by converting the active ingredient(s) with the excipients customary in pharmaceutical technology into the desired administration form.

Excipients which can be employed in this connection are, for example, carrier substances, fillers, disintegrants, binders, humectants, lubricants, absorbents and adsorbents, diluents, solvents, cosolvents, emulsifiers, solubilizers, masking flavours, colorants, preservatives, stabilizers, wetting agents, salts to alter the osmotic pressure or buffers. Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may be, for example, salts, saccharides (mono-, di-, tri-, oligo- and/or polysaccharides), proteins, amino acids, peptides, fats, waxes, oils, hydrocarbons and their derivatives, where the excipients may be of natural origin or may be obtained by synthesis or partial synthesis.

Suitable for oral or peroral administration are in particular tablets, coated tablets, capsules, pills, powders, granules, pastilles, suspensions, emulsions or solutions. Suitable for parenteral administration are in particular suspensions, emulsions and especially solutions.

### Preparation of the Compounds According to Formula (I) According to the Invention

2-Chloropyrimidines of the formula (II) can be reacted with nucleophiles of the formula (III) to give compounds of the formula (I)

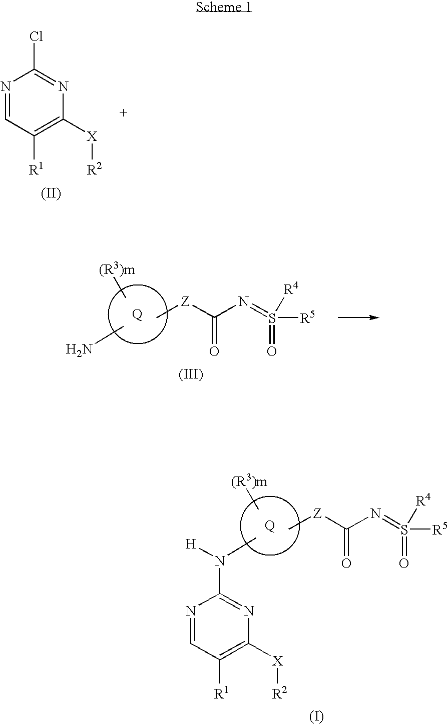

The substituents Q, R¹, R², R³, R⁴, R⁵, X, Z and m have the meanings indicated in the general formula (I).

### Preparation of the Intermediates of the Formula (II):

2,4-Dichloropyrimidines of the formula (V) can be reacted with nucleophiles of the formula (IV) to give compounds of the formula (II) (see, for example: a) U. Lücking et al., WO 2005037800; b) J. Bryant et al., WO 2004048343; c) U. Lücking et al., WO 2003076437; d) T. Brumby et al., WO 2002096888).

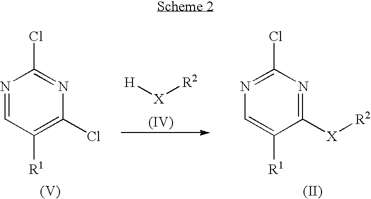

The substituents R¹, R² and X have the meanings indicated in the general formula (I).

### Preparation of the Intermediates of the Formula (III), in Particular of the Formula (IIIa) and (IIIb):

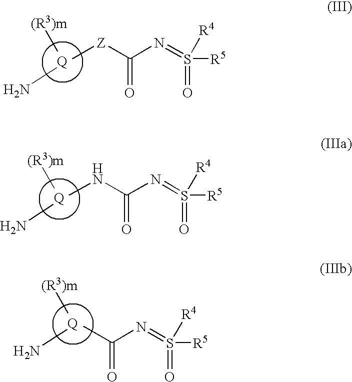

where Q, Z, R³, R⁴ and R⁵ have the meanings indicated in the general formula (I) according to claims 1 to 18.

### Intermediates of the Formula (IIIa):

Isocyanates of the formula (VII) can be reacted with sulphoximines of the formula (VIII) to give intermediates of the formula (VI)

A number of methods are available for the subsequent reduction of the nitro group (see, for example: R. C. Larock, Comprehensive Organic Transformations, VCH, New York, 1989, 411-415). For example, the described hydrogenation using Raney nickel, the use of titanium(III) chloride in THF or the use of palladium on carbon and ammonium formate is suitable.

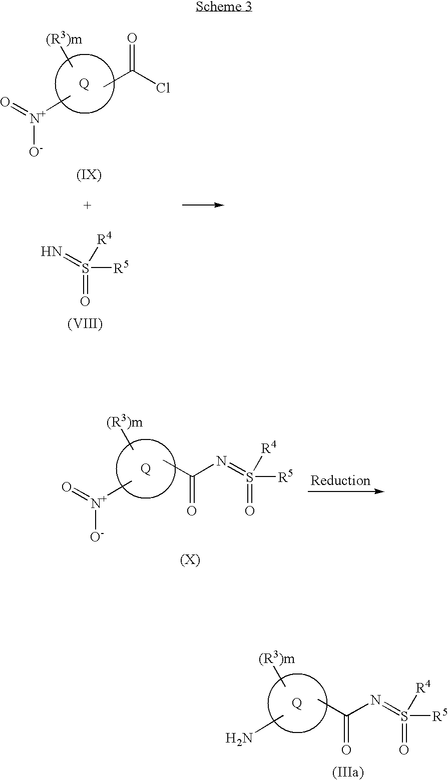

The substituents Q, R³, R⁴, R⁵ and m have the meanings indicated in the general formula (I).

### Preparation of the Intermediates of the Formula (IIIb):

Acid chlorides of the formula (IX) can be reacted with sulphoximines of the formula (VIII) to give intermediates of the formula (X)

A number of methods are available for the subsequent reduction of the nitro group (see, for example: R. C. Larock, Comprehensive Organic Transformations, VCH, New York, 1989, 411-415). For example, the described hydrogenation using Raney nickel, the use of titanium(III) chloride in THF or the use of palladium on carbon and ammonium formate is suitable.

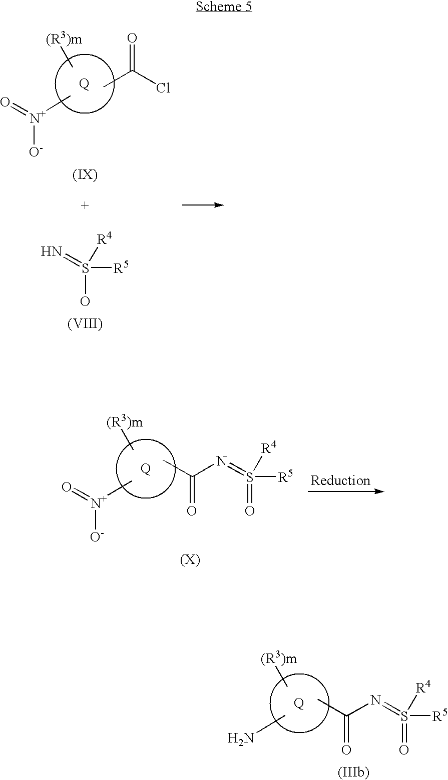

The substituents Q, R³, R⁴, R⁵ and m have the meanings indicated in the general formula (I).

### EXAMPLE 1

### N-({3-[(5-Bromo-4-{[(R)-2-hydroxy-1,2-dimethylpropyl]amino}pyrimidin-2-yl)amino]phenyl}carbamoyl)-S,S-dimethylsulphoximide

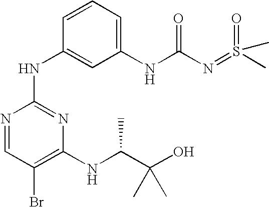

### 1a) Preparation of the Intermediates

### Compound 1.1

### (R)-3-(5-Bromo-2-chloropyrimidin-4-ylamino)-2-methylbutan-2-ol

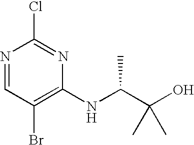

Preparation according to: Lücking et al., WO 2005/037800, page 94.

### Compound 1.2

### S,S-Dimethyl-N-[(3-nitrophenyl)carbamoyl]sulphoximide

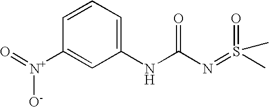

A mixture with 420 mg (4.45 mmol) of dimethylsulphoximine (for preparation, see for example Johnson et al., J. Org. Chem. 1973, 38, 1793) and 730 mg (4.51 mmol) of 3-nitrophenyl isocyanate in 6 ml of acetonitrile is heated to 40° C. After one hour, the mixture is cooled, and the precipitate which has formed is filtered off. The precipitate is washed with acetonitrile and then dried. 667 mg (2.60 mmol; corresponding to 57% of theory) of the product are obtained.

¹H-NMR (DMSO): 9.72 (s, 1H), 8.56 (m, 1H), 7.75 (m, 2H), 7.46 (m, 1H), 3.35 (s, 6H).

MS: 258 (ES).

### Compound 1.3

### N-[(3-Aminophenyl)carbamoyl]S,S-dimethylsulphoximide

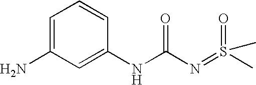

A mixture with 130 mg (0.51 mmol) of S,S-dimethyl-N-[(3-nitrophenyl)carbamoyl]-sulphoximide, 127 mg (2.02 mmol) of ammonium formate, and 10 mg of 10% palladium on carbon in 5 ml of methanol is stirred under argon at room temperature for 3 hours. The mixture is filtered and the filter cake is washed with dichloromethane/methanol (1:1) and methanol. The filtrate is concentrated. 75 mg (0.33 mmol; corresponding to 65% of theory) of the product are obtained.

¹H-NMR (DMSO): 8.83 (s, 1H), 6.80 (m, 2H), 6.57 (m, 1H), 6.10 (m, 1H), 4.84 (m, 2H), 3.28 (s, 6H).

MS: 228 (ES).

### 1b) Preparation of the Final Product

91 mg (0.31 mmol) of (R)-3-(5-bromo-2-chloropyrimidin-4-ylamino)-2-methylbutan-2-ol and 70 mg (0.31 mmol) of N-[(3-aminophenyl)carbamoyl]-S,S-dimethyl-sulphoximide in 5 ml of 1-butanol and 0.5 ml of methanol are stirred at 70° C. for 7 days. After cooling, the mixture is filtered and the filter cake is washed with 1-butanol. The filtrate is concentrated, and the residue formed is purified by chromatography (dichloromethane/ethanol 9:1). 40 mg (0.08 mmol; corresponding to 46% of theory) of the product are obtained.

¹H-NMR (DMSO): 9.02 (m, 2H), 7.96 (s, 1H), 7.86 (m, 1H), 7.21 (m, 1H), 7.01 (m, 1H), 6.91 (m, 1H), 5.90 (d, 1H), 4.70 (s, 1H), 4.11 (m, 1H), 3.30 (s, 6H), 1.13 (m, 9H).

MS: 485 (ES).

### EXAMPLE 2

### N-({4-[(5-Bromo-4-{[(R)-2-hydroxy-1,2-dimethylpropyl]amino}pyrimidin-2-yl)-amino]phenyl}carbamoyl)-S,S-dimethylsulphoximide

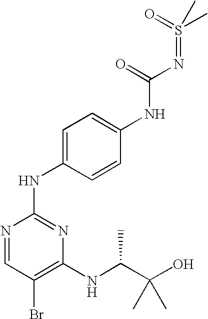

### 2a) Preparation of the Intermediates

### Compound 2.1

### S,S-Dimethyl-N-[(4-nitrophenyl)carbamoyl]sulphoximide

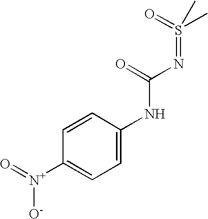

A mixture with 770 mg (8.27 mmol) of dimethylsulphoximine and 1233 mg (7.51 mmol) of 4-nitrophenyl isocyanate in 10 ml of acetonitrile is heated to 45° C. After 3 hours, the mixture is cooled and the precipitate which has formed is filtered off. The precipitate is washed with dichloromethane and then dried. 1840 mg (7.15 mmol; corresponding to 95% of theory) of the product are obtained.

¹H-NMR (DMSO): 9.93 (s, 1H), 8.10 (m, 2H), 7.72 (m, 2H), 3.36 (s, 6H).

MS: 257 (ES).

### Compound 2.2

### N-[(4-Aminophenyl)carbamoyl]S,S-dimethylsulphoximide

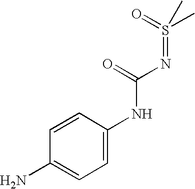

A mixture with 500 mg (1.94 mmol) of S,S-dimethyl-N-[(4-nitrophenyl)carbamoyl]-sulphoximide, 490 mg (7.77 mmol) of ammonium formate and 40 mg of 10% palladium on carbon in 20 ml of methanol is stirred under argon at room temperature for 2 hours. The mixture is filtered and the filter cake is washed with dichloromethane/methanol (1:1) and methanol. The filtrate is concentrated. 417 mg (1.83 mmol; corresponding to 94% of theory) of the product are obtained.

¹H-NMR (DMSO): 8.66 (br, 1H), 7.09 (m, 2H), 6.40 (m, 2H), 4.62 (br, 2H), 3.26 (s, 6H).

MS: 228 (ES).

### 2b) Preparation of the Final Product

100 mg (0.34 mmol) of (R)-3-(5-bromo-2-chloropyrimidin-4-ylamino)-2-methylbutan-2-ol and 70 mg (0.31 mmol) of N-[(4-aminophenyl)carbamoyl]-S,S-dimethyl-sulphoximide in 5 ml of 1-butanol and 0.5 ml of methanol are stirred at 70° C. for 5 days. After cooling, the mixture is filtered and the filter cake is washed with 1-butanol. The filtrate is concentrated, and the residue formed is purified by chromatography (dichloromethane/ethanol 9:1). 60 mg (0.12 mmol; corresponding to 40% of theory) of the product are obtained.

¹H-NMR (DMSO): 8.99 (m, 2H), 7.95 (s, 1H), 7.47 (m, 2H), 7.33 (m, 2H), 5.89 (d, 1H), 4.76 (s, 1H), 4.03 (m, 1H), 3.29 (s, 6H), 1.10 (m, 9H).

MS: 485 (ES).

### EXAMPLE 3

### N-({4-[(5-Bromo-4-{[(1R,2R)-2-hydroxy-1-methylpropyl]amino}pyrimidin-2-yl)amino]phenyl}carbamoyl)-S,S-dimethylsulphoximide

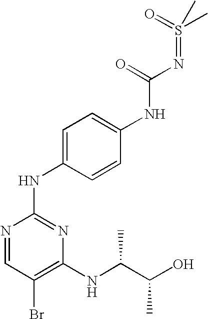

### 3a) Preparation of the Intermediates

### Compound 3.1

### (2R,3R)-3-(5-Bromo-2-chloropyrimidin-4-ylamino)butan-2-ol

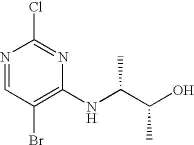

Preparation according to: Lücking et al., WO 2005/037800, page 95.

### 3b) Preparation of the Final Product

104 mg (0.34 mmol) of (2R,3R)-3-(5-bromo-2-chloropyrimidin-4-ylamino)butan-2-ol and 70 mg (0.31 mmol) of N-[(4-aminophenyl)carbamoyl]-S,S-dimethylsulphoximide (compound 2.2) in 5 ml of 1-butanol and 0.5 ml of methanol are stirred at 70° C. for 5 days. The mixture is concentrated and the residue formed is purified by chromatography (dichloromethane/ethanol 9:1). 86 mg (0.18 mmol; corresponding to 59% of theory) of the product are obtained.

¹H-NMR (DMSO): 9.01 (m, 2H), 7.95 (s, 1H), 7.48 (m, 2H), 7.34 (m, 2H), 5.91 (d, 1H), 4.95 (d, 1H), 3.99 (m, 1H), 3.72 (m, 1H), 3.30 (s, 6H), 1.14 (d, 3H), 1.03 (d, 3H).

MS: 471 (ES).

### EXAMPLE 4

### N-({4-[(5-Bromo-4-{[(1R,2R)-2-hydroxy-1-methylpropyl]oxy}pyrimidin-2-yl)amino]phenyl}carbamoyl)-S,S-dimethylsulphoximide

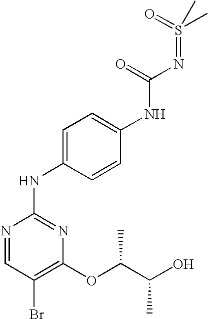

### 4a) Preparation of the Intermediates

### Compound 4.1

### (2R,3R)-3-(5-Bromo-2-chloropyrimidin-4-yloxy)butan-2-ol

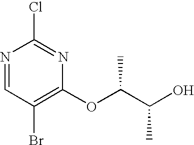

Preparation according to: Lücking et al., WO 2005/037800, page 93.

### 4b) Preparation of the Final Product

104 mg (0.34 mmol) of (2R,3R)-3-(5-bromo-2-chloropyrimidin-4-yloxy)butan-2-ol and 70 mg (0.31 mmol) of N-[(4-aminophenyl)carbamoyl]-S,S-dimethylsulphoximide (compound 2.2) in 5 ml of 1-butanol and 0.5 ml of methanol are stirred at 70° C. for 5 days. After cooling, the mixture is filtered and the filter cake is washed with 1-butanol. The filtrate is concentrated and the residue formed is purified by chromatography (dichloromethane/ethanol 8:2). 20 mg (0.04 mmol; corresponding to 14% of theory) of the product are obtained.

¹H-NMR (DMSO): 9.43 (s, 1H), 9.05 (br, 1H), 8.25 (s, 1H), 7.46 (m, 2H), 7.38 (m, 2H), 5.12 (m, 1H), 4.82 (d, 1H), 3.76 (m, 1H), 3.30 (s, 6H), 1.21 (d, 3H), 1.07 (d, 3H).

MS: 472 (ES).

### EXAMPLE 5

### N-({3-[(5-Bromo-4-{[(1R,2R)-2-hydroxy-1-methylpropyl]amino}pyrimidin-2-yl)amino]phenyl}carbamoyl)-S,S-dimethylsulphoximide

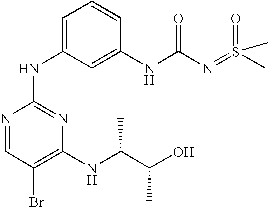

### Preparation of the Final Product

284 mg (1.01 mmol) of (2R,3R)-3-(5-bromo-2-chloropyrimidin-4-ylamino)butan-2-ol (compound 3.1) and 230 mg (1.01 mmol) of N-[(3-aminophenyl)carbamoyl]-S,S-dimethylsulphoximide (compound 1.3) in 16.4 ml of 1-butanol and 1.6 ml of methanol are stirred at 60° C. for 5 days. After cooling, the mixture is filtered and the filter cake is washed with 1-butanol. The filtrate is concentrated and the residue formed is purified by chromatography (dichloromethane/ethanol 8:2). 27 mg (0.06 mmol; corresponding to 6% of theory) of the product are obtained.

¹H-NMR (DMSO): 9.06 (s, 1H), 9.01 (s, 1H), 8.00 (s, 1H), 7.96 (s, 1H), 7.16 (m, 1H), 7.00 (m, 1H), 6.88 (m, 1H), 5.90 (d, 1H), 4.91 (d, 1H), 4.15 (m, 1H), 3.70 (m, 1H), 3.30 (s, 6H), 1.15 (d, 3H), 1.03 (d, 3H).

MS: 471 (ES).

### EXAMPLE 6

### N-({3-[(5-Bromo-4-{[(1R,2R)-2-hydroxy-1-methyl propyl]oxy}pyrimidin-2-yl)amino]phenyl}carbamoyl)-S,S-dimethylsulphoximide

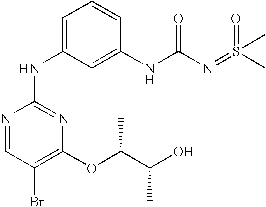

### Preparation of the Final Product

112 mg (0.40 mmol) of (2R,3R)-3-(5-bromo-2-chloropyrimidin-4-yloxy)butan-2-ol (compound 4.1) and 90 mg (0.40 mmol) of N-[(3-aminophenyl)carbamoyl]-S,S-dimethylsulphoximide (compound 1.3) in 7 ml of 1-butanol and 0.7 ml of methanol are stirred at 60° C. for 8 days. After cooling, the mixture is filtered and the filter cake is washed with 1-butanol. The filtrate is concentrated and the residue formed is purified by chromatography (dichloromethane/ethanol 8:2). 59 mg (0.12 mmol; corresponding to 31% of theory) of the product are obtained.

¹H-NMR (DMSO): 9.50 (s, 1H), 9.08 (s, 1H), 8.27 (s, 1H), 7.92 (m, 1H), 7.19 (m, 1H), 7.05 (m, 1H), 6.98 (m, 1H), 5.23 (m, 1H), 4.75 (d, 1H), 3.78 (m, 1H), 3.31 (s, 6H), 1.21 (d, 3H), 1.07 (d, 3H). MS: 472 (ES).

### EXAMPLE 7

### N-({5-[(5-Bromo-4-{[(1R,2R)-2-hydroxy-1-methylpropyl]amino}pyrimidin-2-yl)amino]-2-fluorophenyl}carbamoyl)-S,S-dimethylsulphoximide

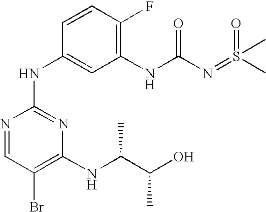

### 7a) Preparation of the Intermediates

### Compound 7.1

### N-[(2-Fluoro-5-nitrophenyl)carbamoyl]-S,S-dimethylsulphoximide

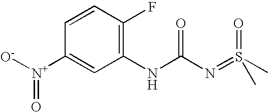

Reaction of 1-fluoro-2-isocyanate-4-nitrobenzene and dimethylsulphoximine in analogy to the method for preparing compound 2.1 afforded the desired product in a yield of 88%.

¹H-NMR (DMSO): 9.28 (br, 1H), 8.84 (m, 1H), 7.87 (m, 1H), 7.42 (m, 1H), 3.36 (s, 6H). MS: 275 (EI).

### Compound 7.2

### N-[(5-Amino-2-fluorophenyl)carbamoyl]-S,S-dimethylsulphoximide

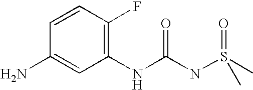

Reduction of N-[(2-fluoro-5-nitrophenyl)carbamoyl]-S,S-dimethylsulphoximide in analogy to the method for preparing compound 2.2 afforded the desired product in a yield of 85%.

¹H-NMR (DMSO): 8.18 (s, 1H), 6.97 (m, 1H), 6.74 (m, 1H), 6.14 (m, 1H), 4.82 (br, 2H), 3.29 (s, 6H).

### 7b) Preparation of the Final Product

154 mg (0.55 mmol) of (2R,3R)-3-(5-bromo-2-chloropyrimidin-4-ylamino)butan-2-ol (compound 3.1) and 122 mg (0.50 mmol) of N-[(5-amino-2-fluorophenyl)carbamoyl]-S,S-dimethylsulphoximide in 8.1 ml of 1-butanol and 0.8 ml of methanol are stirred at 70° C. for 5 days. The mixture is concentrated and the residue formed is purified by chromatography (dichloromethane/ethanol 9:1). 30 mg (0.06 mmol; corresponding to 12% of theory) of the product are obtained.

¹H-NMR (DMSO): 9.15 (s, 1H), 8.40 (s, 1H), 8.12 (m, 1H), 7.97 (s, 1H), 7.24 (m, 1H), 6.98 (m, 1H), 5.90 (d, 1H), 4.91 (d, 1H), 4.14 (m, 1H), 3.72 (m, 1H), 3.31 (s, 6H), 1.14 (d, 3H), 1.03 (d, 3H). MS: 488 (EI).

### EXAMPLE 8

### N-({5-[(5-Bromo-4-{[(1R,2R)-2-hydroxy-1-methylpropyl]amino}pyrimidin-2-yl)amino]-2-methylphenyl}carbamoyl)-S,S-dimethylsulphoximide

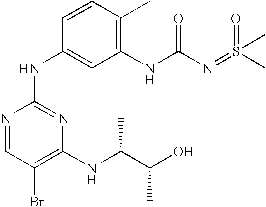

### 8a) Preparation of the Intermediates

### Compound 8.1

### N-[(2-Methyl-5-nitrophenyl)carbamoyl]-S,S-dimethylsulphoximide

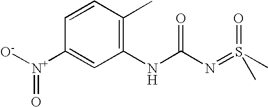

Reaction of 2-isocyanate-1-methyl-4-nitrobenzene and dimethylsulphoximine in analogy to the method for preparing compound 2.1 afforded the desired product in a yield of 88%.

¹H-NMR (DMSO): 8.68 (s, 1H), 8.52 (m, 1H), 7.76 (m, 1H), 7.38 (m, 1H), 3.35 (s, 6H), 2.29 (s, 3H). MS: 271 (EI).

### Compound 8.2

### N-[(5-Amino-2-methylphenyl)carbamoyl]-S,S-dimethylsulphoximide

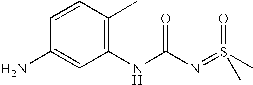

Reduction of N-[(2-methyl-5-nitrophenyl)carbamoyl]-S,S-dimethylsulphoximide in analogy to the method for preparing compound 2.2 afforded the desired product in a yield of 96%.

¹H-NMR (DMSO): 7.91 (s, 1H), 6.72 (m, 2H), 6.16 (m, 1H), 4.71 (br, 2H), 3.30 (s, 6H), 1.96 (s, 3H).

### 8b) Preparation of the Final Product

154 mg (0.55 mmol) of (2R,3R)-3-(5-bromo-2-chloropyrimidin-4-ylamino)butan-2-ol (compound 3.1) and 121 mg (0.50 mmol) of N-[(5-amino-2-methylphenyl)carbamoyl]-S,S-dimethylsulphoximide in 8.1 ml of 1-butanol and 0.8 ml of methanol are stirred at 70° C. for 5 days. After cooling, the precipitate which has formed is filtered off with suction, washed with a little 1-butanol and dried. 60 mg (0.12 mmol; corresponding to 25% of theory) of the product are obtained.

¹H-NMR (DMSO): 9.90 (br, 1H), 8.26 (s, 1H), 8.13 (s, 1H), 7.84 (m, 1H), 7.15 (m, 1H), 7.03 (m, 1H), 4.12 (m, 1H), 3.49 (m, 1H), 3.31 (s, 6H), 2.12 (s, 3H), 1.14 (d, 3H), 1.03 (d, 3H). MS: 485 (ES).

### EXAMPLE 9

### N-({3-[(5-Bromo-4-{[(1R,2R)-2-hydroxy-1-methylpropyl]amino}pyrimidin-2-yl)amino]-2-methylphenyl)carbamoyl)-S,S-dimethylsulphoximide

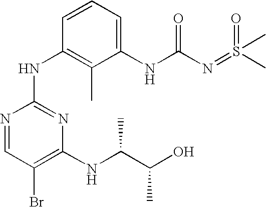

### 9a) Preparation of the Intermediates

### Compound 9.1

### N-[(2-Methyl-3-nitrophenyl)carbamoyl]-S,S-dimethylsulphoximide

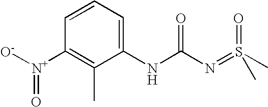

Reaction of 1-isocyanate-2-methyl-3-nitrobenzene and dimethylsulphoximine in analogy to the method for preparing compound 2.1 afforded the desired product in a yield of 79%.

¹H-NMR (DMSO): 8.83 (s, 1H), 7.65 (m, 1H), 7.54 (m, 1H), 7.31 (m, 1H), 3.32 (s, 6H), 2.19 (s, 3H). MS: 271 (ES).

### Compound 9.2

### N-[(3-Amino-2-methylphenyl)carbamoyl]S,S-dimethylsulphoximide

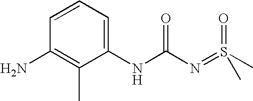

Reduction of N-[(2-methyl-3-nitrophenyl)carbamoyl]-S,S-dimethylsulphoximide in analogy to the method for preparing compound 2.2 afforded the desired product in a yield of 91%.

¹H-NMR (DMSO): 8.14 (s, 1H), 6.73 (m, 1H), 6.51 (m, 1H), 6.36 (m, 1H), 4.71 (br, 2H), 3.30 (s, 6H), 1.84 (s, 3H).

### 9b) Preparation of the Final Product

154 mg (0.55 mmol) of (2R,3R)-3-(5-bromo-2-chloropyrimidin-4-ylamino)butan-2-ol (compound 3.1) and 120 mg (0.50 mmol) of N-[(3-amino-2-methylphenyl)carbamoyl]-S,S-dimethylsulphoximide in 8.1 ml of 1-butanol and 0.8 ml of methanol are stirred at 70° C. for 8 days. The mixture is concentrated and purified by chromatography (dichloromethane/ethanol 9:1). 11 mg (0.02 mmol; corresponding to 5% of theory) of the product are obtained.

¹H-NMR (DMSO): 8.36 (m, 2H), 7.86 (s, 1H), 7.14 (m, 1H), 7.08 (m, 1H), 6.99 (m, 1H), 5.82 (d, 1H), 4.88 (d, 1H), 3.85 (m, 1H), 3.65 (m, 1H), 3.28 (s, 3H), 3.27 (s, 3H), 1.99 (s, 3H), 1.07 (d, 3H), 0.99 (d, 3H).

### EXAMPLE 10

### N-({5-[(5-Bromo-4-{[(1R,2R)-2-hydroxy-1-methylpropyl]amino}pyrimidin-2-yl)amino]-2-methoxyphenyl}carbamoyl)-S,S-dimethylsulphoximide

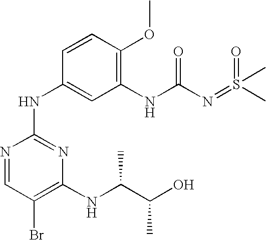

### 10a) Preparation of the Intermediates

### Compound 10.1

### N-[(2-Methoxy-5-nitrophenyl)carbamoyl]-S,S-dimethylsulphoximide

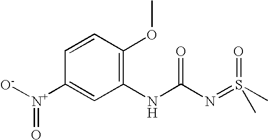

Reaction of 2-isocyanate-1-methoxy-4-nitrobenzene and dimethylsulphoximine in analogy to the method for preparing compound 2.1 afforded the desired product in a yield of 79%.

¹H-NMR (DMSO): 8.87 (s, 1H), 7.90 (m, 2H), 7.16 (m, 1H), 3.92 (s, 3H), 3.35 (s, 6H).

MS: 287 (EI)

### Compound 10.2

### N-[(5-Amino-2-methoxyphenyl)carbamoyl]-S,S-dimethylsulphoximide

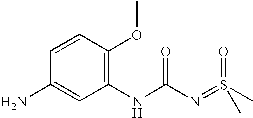

Reduction of N-[(2-methoxy-5-nitrophenyl)carbamoyl]-S,S-dimethylsulphoximide in analogy to the method for preparing compound 2.2 afforded the desired product in a yield of 100%.

¹H-NMR (DMSO): 7.30 (m, 1H), 7.23 (s, 1H), 6.63 (m, 1H), 6.10 (m, 1H), 4.71 (br, 2H), 3.64 (s, 3H), 3.30 (s, 6H).

### 10b) Preparation of the Final Product

154 mg (0.55 mmol) of (2R,3R)-3-(5-bromo-2-chloropyrimidin-4-ylamino)butan-2-ol (compound 3.1) and 128 mg (0.50 mmol) of N-[(3-amino-2-methoxyphenyl)-carbamoyl]-S,S-dimethylsulphoximide in 8.1 ml of 1-butanol and 0.8 ml of methanol are stirred at 70° C. for 5 days. The mixture is concentrated and purified by chromatography (dichloromethane/ethanol 9:1). 53 mg (0.11 mmol; corresponding to 21% of theory) of the product are obtained.

¹H-NMR (DMSO): 9.87 (s, 1H), 8.31 (br, 1H), 8.13 (s, 1H), 7.54 (s, 1H), 7.11 (m, 1H), 6.97 (m, 2H), 4.24 (m, 1H), 3.82 (s, 3H), 3.78 (m, 1H), 3.36 (s, 3H), 3.35 (s, 3H), 1.18 (d, 3H), 1.08 (d, 3H).

MS: 500 (EI).

### EXAMPLE 11

### N-({4-[(5-Bromo-4{[(1R,2R)-2-hydroxy-1-methylpropyl]amino}pyrimidin-2-yl)amino]-2-methoxyphenyl}carbamoyl)-S,S-dimethylsulphoximide

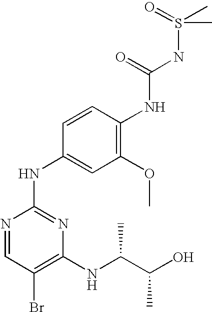

### 11a) Preparation of the Intermediates

### Compound 11.1

### N-[(2-Methoxy-4-nitrophenyl)carbamoyl]-S,S-dimethylsulphoximide

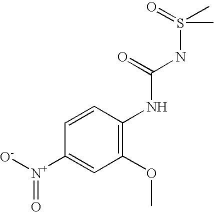

Reaction of 1-isocyanate-2-methoxy-4-nitrobenzene and dimethylsulphoximine in analogy to the method for preparing compound 2.1 afforded the desired product in a yield of 90%.

¹H-NMR (DMSO): 8.24 (m, 1H), 7.96 (s, 1H), 7.86 (m, 1H), 7.72 (m, 1H), 3.92 (s, 3H), 3.36 (s, 6H), MS: 287 (EI)

### Compound 11.2

### N-[(4-Amino-2-methoxyphenyl)carbamoyl]-S,S-dimethylsulphoximide

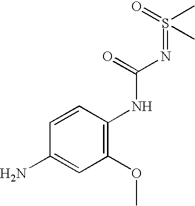

Reduction of N-[(2-methoxy-4-nitrophenyl)carbamoyl]-S,S-dimethylsulphoximide in analogy to the method for preparing compound 2.2 afforded the desired product in a yield of 100%.

¹H-NMR (DMSO): 7.28 (br, 1H), 7.19 (br, 1H), 6.20 (m, 1H), 6.03 (m, 1H), 4.60 (br, 2H), 3.65 (s, 3H), 3.30 (s, 6H).

### 11b) Preparation of the Final Product

154 mg (0.55 mmol) of (2R,3R)-3-(5-bromo-2-chloropyrimidin-4-ylamino)butan-2-ol (compound 3.1) and 128 mg (0.50 mmol) of N-[(4-amino-2-methoxyphenyl)-carbamoyl]-S,S-dimethylsulphoximide in 8.1 ml of 1-butanol and 0.8 ml of methanol are stirred at 70° C. for 5 days. The mixture is concentrated and purified by chromatography (dichloromethane/ethanol 9:1). 41 mg (0.08 mmol; corresponding to 16% of theory) of the product are obtained.

¹H-NMR (DMSO): 9.50 (br, 1H), 8.04 (s, 1H), 7.73 (m, 1H), 7.44 (s, 1H), 7.36 (m, 1H), 7.03 (m, 1H), 6.55 (br, 1H), 4.05 (m, 1H), 3.76 (s, 3H), 3.75 (m, 1H), 3.30 (s, 6H), 1.14 (d, 3H), 1.02 (d, 3H).

MS: 500 (EI).

### EXAMPLE 12

### N-({5-[(5-Bromo-4-{[(1R,2R)-2-hydroxy-1-methylpropyl]amino}pyrimidin-2-yl)amino]-2-chlorophenyl}carbamoyl)-S,S-dimethylsulphoximide

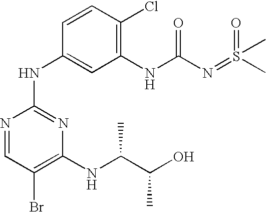

### 12a) Preparation of Intermediates

### Compound 12.1

### N-[(2-Chloro-5-nitrophenyl)carbamoyl]-S,S-dimethylsulphoximide

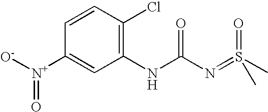

Reaction of 1-chloro-2-isocyanate-4-nitrobenzene and dimethylsulphoximine in analogy to the method for preparing compound 2.1 afforded the desired product in a yield of 92%.

¹H-NMR (DMSO): 8.78 (m, 1H), 8.57 (s, 1H), 7.83 (m, 1H), 7.69 (m, 1H), 3.37 (s, 6H). MS: 291 (ES).

### Compound 12.2

### N-[(5-Amino-2-chlorophenyl)carbamoyl]-S,S-dimethylsulphoximide

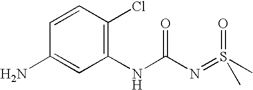

Reduction of N-[(2-chloro-5-nitrophenyl)carbamoyl]-S,S-dimethylsulphoximide in analogy to the method for preparing compound 2.2 and subsequent purification by chromatography (dichloromethane/ethanol 9:1) afforded the desired product in a yield of 13%.

¹H-NMR (DMSO): 7.59 (s, 1H), 7.15 (m, 1H), 6.95 (m, 1H), 6.20 (m, 1H), 5.16 (br, 2H), 3.31 (s, 6H). MS: 261 (EI).

### 12b) Preparation of the Final Product

159 mg (0.57 mmol) of (2R,3R)-3-(5-bromo-2-chloropyrimidin-4-ylamino)butan-2-ol (compound 3.1) and 135 mg (0.52 mmol) of N-[(5-amino-2-chlorophenyl)carbamoyl]-S,S-dimethylsulphoximide in 8.4 ml of 1-butanol and 0.8 ml of methanol are stirred at 70° C. for 4 days. The mixture is cooled to 0° C. The precipitate which has formed is filtered off with suction and washed with cold 1-butanol. Drying results in 178 mg (0.35 mmol; corresponding to 68% of theory) of the product.

¹H-NMR (DMSO): 10.08 (s, 1H), 8.26 (m, 1H), 8.15 (s, 1H), 8.03 (m, 1H), 7.32 (m, 1H), 7.24 (m, 1H), 6.74 (br, 1H), 4.18 (m, 1H), 3.72 (m, 1H), 3.33 (s, 3H), 3.32 (s, 3H), 1.15 (s, 3H), 1.03 (s, 3H).

MS: 504 (E1).

### EXAMPLE 13

### N-[(4-{[4-{[(R)-2-Hydroxy-1-methylethyl]amino}5-(3-thienyl)pyrimidin-2-yl]amino}phenyl)carbamoyl]S,S-dimethylsulphoximide

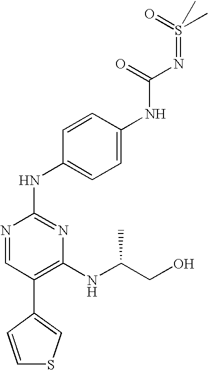

### 13a) Preparation of the Intermediates

### Compound 13.1

### (R)-2-(2-Chloro-5-thiophen-3-yl-pyrimidin-4-ylamino)propan-1-ol

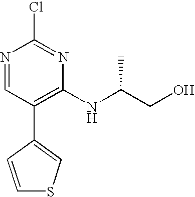

17.3 g (64.8 mmol) of (R)-2-[(5-bromo-2-chloropyrimidin-4-yl)amino]propan-1-ol (for preparation, see: Brumby et al., WO 2002096888, p. 179, Ex. 1-2.42), 9.1 g (71.3 mmol) of thiophene-3-boronic acid, 7.48 g (6.48 mmol) of tetrakis(triphenyl-phosphine)palladium and 1.5 g of tris(2-furyl)phosphine are mixed under argon, and 200 ml of dimethoxyethane are added. This is followed by addition at room temperature of 52 ml of a 2 molar sodium carbonate solution. The mixture is heated to 90° C. and stirred overnight. After cooling, the mixture is mixed with ethyl acetate and washed 3× with water. The organic phase is dried (Na₂SO₄), filtered and concentrated. The resulting residue is purified by chromatography (hexane/ethyl acetate 10-50%). 6.7 g (24.8 mmol; corresponding to 38% of theory) of the product are obtained.

### 13b) Preparation of the Final Product

108 mg (0.40 mmol) of (R)-2-(2-chloro-5-thiophen-3-yl-pyrimidin-4-ylamino)propan-1-ol and 70 mg (0.31 mmol) of N-[(4-aminophenyl)carbamoyl]-S,S-dimethylsulphoxide in 5 ml of 1-butanol and 0.5 ml of methanol are stirred at 70° C. for 5 days. The mixture is concentrated in a rotary evaporator and the residue which has formed is purified by chromatography (dichloromethane/ethanol 9:1). 108 mg (0.23 mmol; corresponding to 76% of theory) of the product are obtained.

¹H-NMR (DMSO): 8.97 (br, 1h), 8.91 (m, 1H), 7.83 (s, 1H), 7.66 (m, 1H) 7.55 (m, 3H), 7.34 (m, 2H), 7.23 (m, 1H), 5.76 (d, 1H), 4.80 (tr, 1H), 4.19 (m, 1H), 3.44 (m, 2H), 3.30 (s, 6H), 1.12 (d, 3H).

MS: 461 (ES).

### EXAMPLE 14

### N-[(3-{[4-{[(R)-2-Hydroxy-1-methylethyl]amino}-5-(3-thienyl)pyrimidin-2-yl]amino}phenyl)carbamoyl]-S,S-dimethylsulphoximide

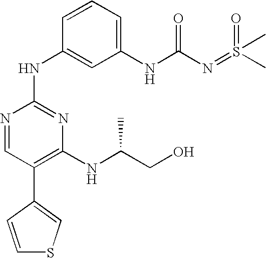

### Preparation of the Final Product

83 mg (0.31 mmol) of (R)-2-(2-chloro-5-thiophen-3-yl-pyrimidin-4-ylamino)propan-1-ol and 70 mg (0.31 mmol) of N-[(3-aminophenyl)carbamoyl]-S,S-dimethylsulphoximide in 5 ml of 1-butanol and 0.5 ml of methanol are stirred at 60° C. for 12 days. After cooling, the mixture is filtered and the filtercake is washed with 1-butanol. The filtrate is concentrated in a rotary evaporator, and the residue which has formed is purified by chromatography (dichloromethane/ethanol 8:2). 60 mg (0.13 mmol; corresponding to 42% of theory) of the product are obtained.

¹H-NMR (DMSO): 9.01 (s, 1H), 8.96 (s, 1H), 7.99 (s, 1H), 7.84 (s, 1H), 7.66 (m, 1H), 7.53 (m, 1H), 7.26 (m, 2H), 7.02 (m, 1H), 6.91 (m, 1H), 5.76 (d, 1H), 4.79 (tr, 1H), 4.32 (m, 1H), 3.45 (m, 2H), 3.30 (s, 6H), 1.11 (d, 3H).

MS: 461 (ES).

### EXAMPLE 15

### N-[(4-{[4-{[1R,2R)-2-Hydroxy-1-methylpropyl]amino}-5-(trifluoro-methyl)pyrimidin-2-yl]amino}phenyl)carbamoyl]-S,S-dimethylsulphoximide

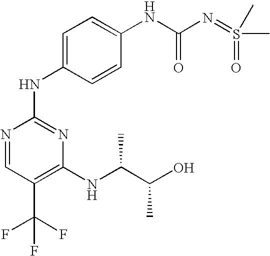

### 15a) Preparation of the Intermediates

### Compound 15.1

### (2R,3R)-3-(2-chloro-5-trifluoromethyl-pyrimidin-4-ylamino)-butan-2-ol

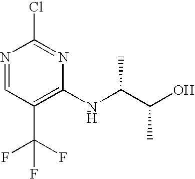

4.8 ml (34.8 mmol) of triethylamine are added dropwise to 3.78 g (17.4 mmol) of 2,4-dichloro-5-trifluoromethylpyrimidine and 2.19 g (17.4 mmol) of (2R,3R)-3-amino-butan-2-ol hydrochloride in 70 ml of acetonitrile at 0° C. The mixture is slowly warmed to room temperature and is then stirred for 48 hours. The mixture is put into a half-concentrated NaCl solution and extracted with ethyl acetate. The combined organic phases are dried (Na₂SO₄), filtered and concentrated. The resulting residue is purified by HPLC. 1.45 g (5.4 mmol; 31% yield) of the product are obtained.

| Column: | XBridge C18 5μ |
|---|---|
| Length × ID: | 100 × 30 mm |
| Eluents: | A: H₂O B: Acetonitrile |
| Buffer: | A/0.1% TFA |
| Gradient: | 60% A + 40% B(2′)_40 −> 70% B(10′) −> 99% B(0.5′) |
| Flow rate: | 40.0 ml/min |
| Detection: | DAD (210-500 nm) TAC; MS-ESI+ (125-800 m/z) TIC |
| Temperature: | Room temperature |
| RT in min: | 5.0-6.0 |

### 15b) Preparation of the Final Product

68 mg (0.25 mmol) of (2R,3R)-3-(2-chloro-5-trifluoromethyl-pyrimidin-4-ylamino)-butan-2-ol and 51 mg (0.22 mmol) of N-[(4-aminophenyl)carbamoyl]-S,S-dimethyl-sulphoximide in 1.8 ml of 1-butanol and 0.2 ml of methanol are stirred at 50° C. for 5 days. The mixture is filtered with suction, and the precipitate which has formed is washed with 1-butanol and MTBE. 60 mg (0.12 mmol; corresponding to 48% of theory) of the product are obtained.

¹H-NMR (DMSO): 10.14 (br, 1H), 9.20 (s, 1H), 8.24 (br, 1H), 7.45 (m, 4H), 6.75 (br, 1H), 4.08 (m, 1H), 3.74 (m, 1H), 3.31 (s, 6H), 1.14 (d, 3H), 1.02 (d, 3H).

MS: 461 (ES).6

### EXAMPLE 16

### N-[(4-{[4-(1H-Benzimidazol-5-ylamino)-5-bromopyrimidin-2-yl]amino}phenyl)-carbamoyl]-S,S-dimethylsulphoximide

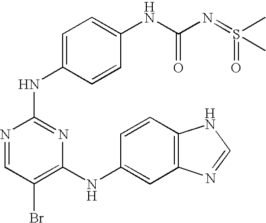

### 16a) Preparation of the Intermediates

### Compound 16.1

### (1H-Benzoimidazol-5-yl)-(5-bromo-2-chloro-pyrimidin-4-yl)-amine

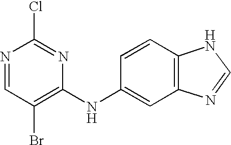

4.31 g (40.6 mmol) of sodium carbonate are added to a solution of 4.51 g (33.9 mmol) of 3H-benzoimidazol-5-ylamine and 9.26 g (40.6 mmol) of 5-bromo-2,4-dichloropyrimidine in 90 ml of ethanol while cooling in water, and the mixture is stirred at room temperature for 24 hours. The mixture is filtered with suction, and the filtercake is washed with ethanol and water and then dried. 10.26 g (31.6 mmol; corresponding to 93% of theory) of the product are obtained.

MS: 325 (EI+).

### 16b) Preparation of the Final Product

111 mg (0.34 mmol) of (1H-benzoimidazol-5-yl)-(5-bromo-2-chloropyrimidin-4-yl)amine and 70 mg (0.31 mmol) of N-[(4-aminophenyl)carbamoyl]-S,S-dimethyl-sulphoximide in 5.0 ml of 1-butanol and 0.5 ml of methanol are stirred at 80° C. for 5 days. The mixture is concentrated in a rotary evaporator and the resulting residue is purified by chromatography (dichloromethane/ethanol 8:2). 32 mg (0.06 mmol; corresponding to 20% of theory) of the product are obtained.

MS: 515 (ESI+)

### EXAMPLE 17

### N-{3-[(5-Bromo-4-{[(1R,2R)-2-hydroxy-1-methylpropyl]amino}pyrimidin-2-yl)amino]benzoyl}-S,S-dimethylsulphoximide

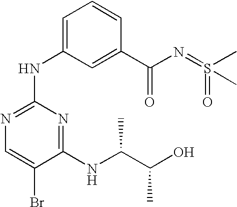

### 17a) Preparation of the Intermediates

### Compound 17.1

### S,S-Dimethyl-N-(3-nitrobenzoyl)sulphoximide

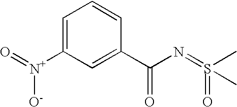

1.1 ml of triethylamine and 1510 mg (8.14 mmol) of 3-nitrobenzoyl chloride are added to a mixture of 510 mg (5.48 mmol) of dimethylsulphoximine (for preparation see, for example, Johnson et al, J. Org. Chem. 1973, 38, 1793) in 30 ml of dichloromethane at room temperature. The mixture is stirred at 40° C. overnight. After cooling, the solid which has formed is filtered off with suction, washed with a little dichloromethane and water and then dried. 350 mg (1.44 mmol; corresponding to 26% of theory) of the product are obtained.

¹H-NMR (DMSO): 8.67 (m, 1H), 8.36 (m, 2H), 7.73 (m, 1H), 3.48 (s, 6H).

MS: 243 (ESI+).

### Compound 17.2

### N-(3-Aminobenzoyl)-S,S-dimethylsulphoximide

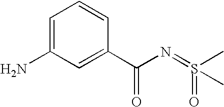

A mixture of 150 mg (0.62 mmol) of S,S-dimethyl-N-(3-nitrobenzoyl)sulphoximide, 156 mg (2.48 mmol) of ammonium formate and 40 mg of 10% palladium on carbon in 20 ml of methanol is stirred under argon at room temperature for 16 hours. The mixture is filtered and the filtercake is washed with dichloromethane/methanol (1:1) and methanol. The filtrate is concentrated in a rotary evaporator. 120 mg (0.57 mmol; corresponding to 91% of theory) of the product are obtained.

¹H-NMR (DMSO); 7.19 (m, 1H), 7.10 (m, 1H), 7.01 (m, 1H), 6.66 (m, 1H), 5.16 (s, 2H), 3.37 (s, 6H).

MS: 213 (ESI+).

### 17b) Preparation of the Final Product

174 mg (0.62 mmol) of ((2R,3R)-3-(5-bromo-2-chloropyrimidin-4-ylamino)butan-2-ol (Compound 3.1) and 120 mg (0.57 mmol) of N-(3-aminobenzoyl)-S,S-dimethyl-sulphoximide in 5.0 ml of 1-butanol and 0.5 ml of methanol are stirred at 70° C. for 5 days. The mixture is filtered with suction and the filtrate is evaporated to dryness. The resulting residue is purified by chromatography (dichloromethane/ethanol 9:1). 44 mg (0.10 mmol; corresponding to 17% of theory) of the product are obtained.

¹H-NMR (DMSO): 9.68 (s, 1H), 8.33 (br, 1H), 8.08 (s, 1H), 7.73 (m, 1H), 7.59 (m, 1H), 7.31 (m, 1H), 6.52 (br, 1H), 4.08 (m, 1H), 3.74 (m, 1H), 3.41 (s, 6H), 1.14 (d, 3H), 1.02 (d, 3H).

MS: 456 (ESI).

### EXAMPLE 18

### N-{4-[(5-Bromo-4-{[(1R,2R)-2-hydroxy-1-methylpropyl]amino}pyrimidin-2-yl)amino]benzoyl}-S,S-dimethylsulphoximide

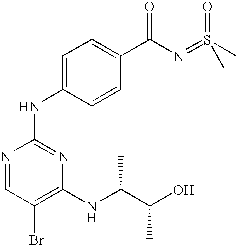

### 18a) Preparation of the Intermediates

### Compound 18.1

### S,S-dimethyl-N-(4-nitrobenzoyl)sulphoximide

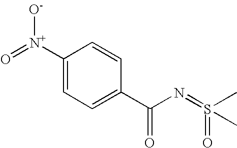

1.1 ml of triethylamine and 1484 mg (8.00 mmol) of 4-nitrobenzoyl chloride are added to a mixture of 500 mg (5.37 mmol) of dimethylsulphoximine (for preparation see, for example, Johnson et al, J. Org. Chem. 1973, 38, 1793) in 30 ml of dichloromethane at room temperature. The mixture is stirred at 40° C. overnight and then cooled to 0° C. The solid which has formed is filtered off with suction, washed with a little dichloromethane and water and then dried. 905 mg (3.74 mmol; corresponding to 70% of theory) of the product are obtained.

¹H-NMR (DMSO): 8.26 (m, 2H), 8.16 (m, 2H), 3.47 (s, 6H).

MS: 242 (EI+).

### Compound 18.2

### N-(4-aminobenzoyl)-S,S-dimethylsulphoximide

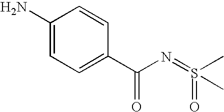

A mixture of 900 mg (3.72 mmol) of S,S-dimethyl-N-(4-nitrobenzoyl)sulphoximide, 940 mg (14.86 mmol) of ammonium formate and 75 mg of 10% palladium on carbon in 75 ml of methanol is stirred at room temperature under argon for 4 hours. The mixture is filtered and the filtercake is subsequently washed with dichloromethane/methanol (1:1) and methanol. The filtrate is concentrated in a rotary evaporator. 759 mg (3.58 mmol; corresponding to 96% of theory) of the product are obtained.

¹H-NMR (DMSO): 7.64 (m, 2H), 6.47 (m, 2H), 5.67 (s, 2H), 3.34 (s, 6H).

MS: 212 (EI+).

### 18b) Preparation of the Final Product

154 mg (0.55 mmol) of ((2R,3R)-3-(5-bromo-2-chloropyrimidin-4-ylamino)butan-2-ol (compound 3.1) and 106 mg (0.57 mmol) of N-(4-aminobenzoyl)-S,S-dimethyl-sulphoximide in 8.1 ml of 1-butanol and 0.8 ml of methanol are stirred at 70° C. for 9 days. The mixture is cooled to 0° C. and filtered with suction. The filtercake is washed with a little 1-butanol and dried. 106 mg (0.23 mmol; corresponding to 46% of theory) of the product are obtained.

¹H-NMR (DMSO): 10.08 (br, 1H), 8.16 (s, 1H), 7.89 (m, 2H), 7.70 (m, 2H), 6.76 (br, 1H), 4.06 (m, 1H), 3.76 (m, 1H), 3.40 (s, 6H), 1.16 (d, 3H), 1.05 (d, 3H).

MS: 457 (EI+).

### EXAMPLE 19

### N-{4-[(5-Bromo-4-{[(R)-2-hydroxy-1,2-dimethylpropyl]amino}pyrimidin-2-yl)amino]benzoyl}S,S-dimethylsulphoximide

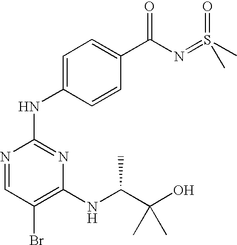

### Preparation of the Final Product

162 mg (0.55 mmol) of (R)-3-(5-bromo-2-chloropyrimidin-4-ylamino)-2-methylbutan-2-ol and 106 mg (0.50 mmol) of N-(4-aminobenzoyl)-S,S-dimethylsulphoximide in 8.1 ml of 1-butanol and 0.8 ml of methanol are stirred at 70° C. for 2 days. The mixture is cooled to 0° C. and filtered with suction. The filtercake is washed with a little 1-butanol and dried. 157 mg (0.33 mmol; corresponding to 67% of theory) of the product are obtained.

¹H-NMR (DMSO): 10.15 (br, 1H), 8.19 (s, 1H), 7.90 (m, 2H), 7.69 (m, 2H), 6.65 (br, 1H), 4.06 (m, 1H), 3.40 (s, 6H), 1.14 (m, 9H).

MS: 471 (EI+).

### EXAMPLE 20

### N-{4-[(5-Bromo-4-{[(1R,2R)-2-hydroxy-1-methylpropyl]oxy}pyrimidin-2-yl)amino]benzoyl}-S,S-dimethylsulphoximide

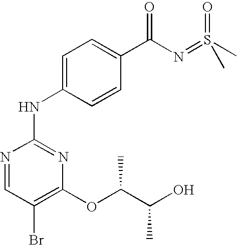

### Preparation of the Final Product

155 mg (0.55 mmol) of ((2R,3R)-3-(5-bromo-2-chloropyrimidin-4-yloxy)butan-2-ol (compound 4.1) and 106 mg (0.50 mmol) of N-(4-aminobenzoyl)-S,S-dimethyl-sulphoximide in 8.1 ml of 1-butanol and 0.8 ml of methanol are stirred at 70° C. for 7 days. The mixture is cooled to 0° C. and filtered with suction. The filtercake is washed with a little 1-butanol and dried. 167 mg (0.37 mmol; corresponding to 73% of theory) of the product are obtained.

¹H-NMR (DMSO): 9.96 (s, 1H), 8.37 (s, 1H), 7.88 (m, 2H), 7.71 (m, 2H), 5.17 (m, 1H), 3.80 (m, 1H), 3.40 (s, 6H), 1.24 (d, 3H), 1.08 (d, 3H).

MS: 471 (EI+).

### EXAMPLE 21

### N-(4-{[4-{[(R)-2-Hydroxy-1-methylethyl]amino}-5-(3-thienyl)pyrimidin-2-yl]amino}benzoyl)-S,S-dimethylsulphoximide

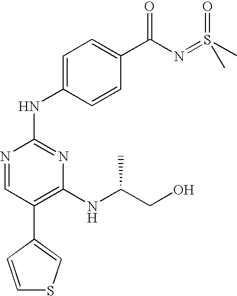

### Preparation of the Final Product

108 mg (0.40 mmol) of (R)-2-(2-chloro-5-thiophen-3-ylpyrimidin-4-ylamino)propan-1-ol and 66 mg (0.31 mmol) of N-(4-aminobenzoyl)-S,S-dimethylsulphoximide in 5.0 ml of 1-butanol and 0.5 ml of methanol are stirred at 70° C. for 3 days. The mixture is cooled to 0° C. and filtered with suction. The filtercake is washed with a little 1-butanol and diisopropyl ether and dried. 25 mg (0.06 mmol; corresponding to 18% of theory) of the product are obtained.

¹H-NMR (DMSO): 10.87 (s, 1H), 7.96 (m, 3H), 7.71 (m, 4H), 7.49 (d, 1H), 7.24 (m, 1H), 4.26 (m, 1H), 3.50 (m, 2H), 3.42 (s, 6H), 1.15 (d, 3H).

MS: 446 (ES+).

### EXAMPLE 22

### N-(4-{[4-(1H-Benzimidazol-5-ylamino)-5-bromopyrimidin-2-yl]amino}benzoyl)-S,S-dimethylsulphoximide

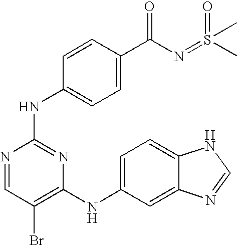

### Preparation of the Final Product

178 mg (0.55 mmol) of (1H-benzoimidazol-5-yl)-(5-bromo-2-chloropyrimidin-4-yl)amine and 106 mg (0.5 mmol) of N-(4-aminobenzoyl)-S,S-dimethylsulphoximide in 8.0 ml of 1-butanol and 0.8 ml of methanol are mixed with 0.026 ml of a 4N solution of hydrogen chloride in dioxane and stirred at 70° C. for 3 days. The mixture is cooled to room temperature and filtered with suction. The filtrate is concentrated and the resulting residue is purified by chromatography (dichloromethane/ethanol 9:1). 16 mg (0.03 mmol; corresponding to 6% of theory) of the product are obtained.

¹H-NMR (DMSO); 9.68 (s, 1H), 9.43 (s, 1H), 9.08 (s, 1H), 8.29 (s, 1H), 7.96 (m, 1H), 7.82 (m, 1H), 7.73 (m, 1H), 7.61 (m, 2H), 7.54 (m, 2H), 3.38 (s, 6H).

MS: 500 (ES+).

### Assay 1

### Aurora-C Kinase Assay

The Aurora-C inhibitory activity of the substances of this invention was measured in the Aurora-C-HTRF assay (HTRF=Homogeneous Time Resolved Fluorescence) described in the following paragraphs.

Recombinant fusion protein of GST and human Aurora-C was expressed in transiently transfected HEK293 cells and purified by affinity chromatography on glutathione-Sepharose. The substrate used for the kinase reaction was the biotinylated peptide biotin-Ttds-FMRLRRLSTKYRT (C terminus in amide form) which can be purchased for example from JERINI Peptide Technologies (Berlin). Aurora-C was incubated in the presence of various concentrations of test substances in 5 μL of assay buffer [25 mM Hepes/NaOH pH 7.4, 0.5 mM MnCl₂, 2.0 mM dithiothreitol, 0.1 mM sodium orthovanadate, 10 μM adenosine triphosphate (ATP), 0.5 μM/ml substrate, 0.01% (v/v) TritonX-100 (Sigma), 0.05% (w/v) bovine serum albumin (BSA), 1% (v/v) dimethyl sulphoxide] at 22° C. for 60 min. The concentration of Aurora-C was adapted to the particular activity of the enzyme and adjusted so that the assay operated in the linear range. Typical concentrations were in the region of 0.3 nM. The reaction was stopped by adding 5 μl of a solution of HTRF detection reagents (0.2 μM streptavidin-XLent and 1.4 nM anti-phospho-(Ser/Thr)-Akt substrate-Eu-cryptate (Cis biointernational, France, product No. 61P02KAE), a Europium-cryptate-labelled phospho-(Ser/Thr)-Akt substrate antibody [product #9611B, Cell Signaling Technology, Danvers, Mass., USA]) in aqueous EDTA solution (40 mM EDTA, 400 mM KF, 0.05% (w/v) bovine serum albumin (BSA) in 25 mM HEPES/NaOH pH 7.0).

The resulting mixture was incubated at 22° C. for 1 h in order to allow formation of a complex of the biotinylated phosphorylated substrate and the detection reagents. The amount of phosphorylated substrate was then estimated by measuring the resonance energy transfer from the anti-phospho-(Ser/Thr)-Akt substrate-Eu cryptate to the streptavidin-XLent. For this purpose, the fluorescence emissions at 620 nm and 665 nm after excitation at 350 nm were measured in an HTRF measuring instrument, e.g. a Rubystar (BMG Labtechnologies, Offenburg, Germany) or a Viewlux (Perkin-Elmer). The ratio of the emissions at 665 nm and at 622 nm was taken as a measure of the amount of phosphorylated substrate. The data were normalized (enzymic reaction without inhibitor=0% inhibition, all other assay components but no enzyme=100% inhibition) and IC50 values were calculated with a 4-parameter fit using an inhouse software.

### Assay 2

### CDK1/CycB Kinase Assay

Recombinant CDK1- and CycB-GST fusion proteins, purified from baculovirus-infected insect cells (Sf9), were purchased from ProQinase GmbH, Freiburg. The histone IIIS used as kinase substrate can be purchased from Sigma.

CDK1/CycB (5 ng/μL) was incubated in the presence of various concentrations of test substances (0 μM, and within the range 0.01-100 μM) in 40 μL of assay buffer [50 mM Tris/HCl pH 8.0, 10 mM MgCl₂, 0.1 mM Na ortho-vanadate, 1.0 mM dithiothreitol, 0.025% PEG 20000, 0.5 μM ATP, 10 μM histone IIIS, 0.2 μCi/measurement point ³³P-gamma ATP, 0.05% NP40, 1.25% dimethyl sulphoxide] at 22° C. for 10 min. The reaction was stopped by adding EDTA solution (250 mM, pH 8.0, 15 μl/measurement point). 15 μl of each reaction mixture were loaded onto P30 filter strips (from Wallac), and non-incorporated ³³P-ATP was removed by washing the filter strips three times in 0.5% strength phosphoric acid for 10 min each time. After the filter strips had been dried at 70° C. for 1 hour, the filter strips were covered with scintillator strips (MeltiLex™ A, from Wallac) and baked at 90° C. for 1 hour. The amount of incorporated ³³P (substrate phosphorylation) was determined by scintillation measurement in a gamma radiation counter (Wallac).

The measured data were normalized to 0% inhibition (enzyme reaction without inhibitor) and 100% inhibition (all assay components except enzyme). The IC50 values were determined by means of a 4-parameter fit using the company's own software.

### Assay 3

### CDK2/CycE Kinase Assay

Recombinant CDK2- and CycE-GST fusion proteins, purified from baculovirus-infected insect cells (Sf9), were purchased from ProQinase GmbH, Freiburg. The histone IIIS used as kinase substrate was purchased from Sigma. CDK2/CycE (1.25 ng/μL) was incubated in the presence of various concentrations of test substances (0 μM, and within the range 0.01-100 μM) in 40 μL of assay buffer [50 mM Tris/HCl pH 8.0, 10 mM MgCl₂, 0.1 mM Na ortho-vanadate, 1.0 mM dithiothreitol, 0.5 μM ATP, 0.2% PEG20000, 10 μM histone IIIS, 0.2 μCi/measurement point ³³P-gamma ATP, 0.05% NP40, 1.25% dimethyl sulphoxide] at 22° C. for 10 min. The reaction was stopped by adding EDTA solution (250 mM, pH 8.0, 15 μl/measurement point).

15 μl of each reaction mixture were loaded onto P30 filter strips (from Wallac), and non-incorporated ³³P-ATP was removed by washing the filter strips three times in 0.5% strength phosphoric acid for 10 min each time.

After the filter strips had been dried at 70° C. for 1 hour, the filter strips were covered with scintillator strips (MeltiLex™ A, from Wallac) and baked at 90° C. for 1 hour. The amount of incorporated ³³P (substrate phosphorylation) was determined by scintillation measurement in a gamma radiation counter (Wallac).

The measured data were normalized to 0% inhibition (enzyme reaction without inhibitor) and 100% inhibition (all assay components except enzyme). The IC50 values were determined by means of a 4-parameter fit using the company's own software.

### Assay 4

### KDR Kinase Assay

Recombinant KDR kinase-GST fusion proteins purified from baculovirus-infected insect cells (Sf9), were purchased from ProQinase GmbH, Freiburg. Poly(Glu₄Tyr)ₙ, which was used as kinase substrate, was purchased from Sigma.

KDR kinase was incubated in the presence of various concentrations of test substances (0 μM, and within the range 0.01-100 μM) in 40 μL of assay buffer [40 mM Tris/HCl pH 7.5, 10 mM MgCl₂, 1 mM MnCl₂, 1.0 mM dithiothreitol, 8 μM ATP, 0.025% PEG20000, 24 ng/μL poly(Glu₄Tyr)ₙ, 0.2 μCi/measurement point ³³P-gamma ATP, 1.25% dimethyl sulphoxide] at 22° C. for 10 min. The reaction was stopped by adding EDTA solution (250 mM, pH 7.5, 15 μl/measurement point). 15 μl of each reaction mixture were loaded onto P30 filter strips (from Wallac), and non-incorporated ³³P-ATP was removed by washing the filter strips three times in 0.5% strength phosphoric acid for 10 min each time.

After the filter strips had been dried at 70° C. for 1 hour, the filter strips were covered with scintillator strips (MeltiLex™ A, from Wallac) and baked at 90° C. for 1 hour. The amount of incorporated ³³P (substrate phosphorylation) was determined by scintillation measurement in a gamma radiation counter (Wallac).

The measured data were normalized to 0% inhibition (enzyme reaction without inhibitor) and 100% inhibition (all assay components except enzyme). The IC50 values were determined by means of a 4-parameter fit using the company's own software.

### Assay 5

### MCF7 Proliferation Assay

Cultivated human MCF7 breast tumour cells (ATCC HTB-22) were plated out in a density of 5000 cells/measurement point in 200 μl of growth medium (RPMI1640, 10% foetal calf serum, 2 mU/mL insulin, 0.1 nM oestradiol) in a 96-well multititre plate. After 24 hours, the cells from a plate (zero plate) were stained with crystal violet (see below), while the medium in the other plates was replaced by fresh culture medium (200 μl) to which the test substances had been added in various concentrations (0 μM, and in the range 0.01-30 μM; the final concentration of the solvent dimethyl sulphoxide was 0.5%). The cells were incubated in the presence of the test substances for 4 days. The cell proliferation was determined by staining the cells with crystal violet: the cells were fixed by adding 20 μl/measurement point of an 11% strength glutaraldehyde solution at room temperature for 15 min. After the fixed cells had been washed three times with water, the plates were dried at room temperature. The cells were stained by adding 100 μl/measurement point of a 0.1% strength crystal violet solution (pH adjusted to pH 3 by adding acetic acid). After the stained cells had been washed three times with water, the plates were dried at room temperature. The dye was dissolved by adding 100 μl/measurement point of a 10% strength acetic acid solution, and the extinction was determined by photometry at a wavelength of 595 nm. The percentage change in cell growth was calculated by normalizing the measurements to the extinctions of the zero point plate (=0%) and the extinction of the untreated (0 μM) cells (=100%). The IC50 values were determined by means of a 4-parameter fit using the company's own software.

### EXAMPLE 23

The compounds of Examples 1 to 22 were tested for their inhibitory effect in the various kinase assays and in a proliferation assay with MCF7 human breast tumour cells (Tab. 1). The data proved that the exemplary compounds act as potent, nanomolar protein kinase inhibitors. Selectivity profiles can be adjusted by varying the substitution pattern (Examples 9, 15, 17: CDK-selective, Example 16: preferential KDR inhibition). Exemplary compounds 1-8, 10-12, 15, 17-20 inhibit the proliferation of human MCF7 breast tumour cells with half-maximum concentrations in the submicromolar range. These data confirm a potential of the carbamoyl- and carbonylsulphoximides for use as tumour therapeutic agents.

TABLE 1

| | | | | KDR | |
|---|---|---|---|---|---|
| | Aurora C | CDK1/CycB | CDK2/CycE | (VEGFR2) | MCF7 |
| Ex. | IC₅₀ [nM] | IC₅₀ [nM] | IC₅₀ [nM] | IC₅₀ [nM] | IC₅₀ [nM] |
| 1 | 160 | 24 | 9 | 170 | 400 |
| 2 | 400 | 21 | 10 | 230 | 500 |
| 3 | 280 | 7 | 5 | 120 | 160 |
| 4 | 420 | 5 | 5 | 160 | 140 |
| 5 | 54 | 10 | 5 | 63 | 160 |
| 6 | 73 | 7 | 7 | 130 | 150 |
| 7 | 370 | 10 | 3 | 300 | 500 |
| 8 | 810 | 21 | 12 | 130 | 700 |
| 9 | 20 000 | 180 | 43 | >1000 | |
| 10 | 410 | 26 | 20 | 320 | 650 |
| 11 | 430 | 46 | 19 | 96 | 990 |
| 12 | 1300 | 18 | 9 | 350 | 860 |
| 15 | 150 | 15 | 9 | 1100 | 530 |
| 16 | 400 | 140 | 330 | 64 | 2100 |
| 17 | 2000 | 10 | 9 | 330 | 380 |
| 18 | 280 | 18 | 9 | 61 | 140 |
| 19 | 740 | 42 | 11 | 120 | 250 |
| 20 | 310 | 17 | 12 | 96 | 310 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102006041382.2, filed Aug. 29, 2006, and U.S. Provisional Application Ser. No. 60/841,567, filed Sep. 1, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula (I), 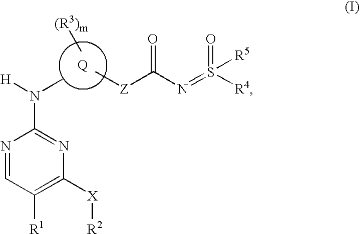
in which
R¹ is
(i) hydrogen, halogen, cyano, nitro, —NR⁸R⁹, —NR⁷—C(O)—R¹², —NR⁷—C(O)—OR¹², —NR⁷—C(O)—NR⁸R⁹, —NR⁷—SO₂—R², —CF₃ or —OCF₃, or
(ii) a C₁-C₆-alkyl, C₂-C₆-alkenyl, C₁-C₆-alkoxy or C₂-C₆-alkynyl radical which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, C₁-C₆-alkoxy, —CF₃ and/or —OCF₃, or
(iii) a phenyl or monocyclic heteroaryl ring which is optionally substituted one or more times, identically or differently, by hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, halogen, —CF₃, C₁-C₆-alkoxy, —OCF₃ and/or C₁-C₆-alkyl,
R² is
(i) hydrogen or
(ii) a C₁-C₁₀-alkyl, C₂-C₁₀-alkenyl or C₂-C₁₀-alkynyl radical, a C₃-C₇-cycloalkyl, phenyl or naphthyl ring, a heterocyclyl ring having 3 to 8 ring atoms or a mono- or bicyclic heteroaryl ring,
in each case optionally substituted one or more times, identically or differently, by
a) halogen, hydroxy, —NR⁸R⁹, —NR⁷—C(O)—R¹², —NR⁷—C(O)—OR¹², —NR⁷—C(O)—NR⁸R⁹, —NR⁷—SO₂—R¹², cyano, —C(O)R⁶, —O(CO)—R¹², —SO₂NR⁸R⁹, —SO₂—R¹², —S(O)(NR⁸)R¹², —(N)S(O)R¹³R¹⁴, —CF₃, —OCF₃, —N[(CO)—(C₁-C₆-alkyl)]₂ and/or
b) C₁-C₆-alkoxy, C₁-C₆-alkyl, C₂-C₆-alkenyl, C₂-C₆-alkynyl, C₃-C₈-cycloalkyl, phenyl, naphthyl, heterocyclyl having 3 to 8 ring atoms and/or a monocyclic or bicyclic heteroaryl, in each case optionally themselves substituted one or more times, identically or differently, by halogen, hydroxy, a C₁-C₆-alkyl, C₁-C₆-alkoxy, —NR⁸R⁹, —C(O)OR¹⁶, —SO₂NR⁸R⁹, —CF₃ or —OCF₃,
R³ is
(i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₆-alkyl and/or C₁-C₆-alkoxy radical which is optionally substituted one or more times, identically or differently, by halogen, hydroxy, C₁-C₆-alkoxy, —CF₃, —OCF₃ or —NR⁸R⁹, and/or
(iii) a C₃-C₇-cycloalkyl ring which is optionally substituted one or more times, identically or differently, by halogen, hydroxy, C₁-C₆-alkoxy, —CF₃, —OCF₃, —NR⁸R⁹ and/or C₁-C₆-alkyl,
m is 0-4,
Z is the group —NH— or a direct linkage,
R⁴ and R⁵ are independently of one another a C₁-C₆-alkyl, C₂-C₆-alkenyl, C₂-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl, or
R⁴ and R⁵ together with the sulphur form a 3 to 7-membered ring which is optionally substituted one or more times, identically or differently, by hydroxy, C₁-C₆-alkyl, C₁-C₆-alkoxy, halogen or —NR⁸R⁹, and optionally comprises a double bond,
X is —O—, —S— or —NR¹⁵—,
where
R¹⁵ is
(i) hydrogen or
(ii) a C₁-C₆-alkyl radical, C₃-C₈-cycloalkyl or phenyl ring, a heterocyclyl ring having 3 to 8 ring atoms or a monocyclic heteroaryl ring, or
(iii) —C(O)—(C₁-C₆)-alkyl, —C(O)-phenyl, or —C(O)-benzyl, and (ii) and (iii) are optionally substituted one or more times, identically or differently, by hydroxy, —NR¹⁰R¹¹, cyano, halogen, —CF₃, C₁-C₆-alkoxy and/or —OCF₃, or
if X is —NR¹⁵—, alternatively —NR¹⁵— and R² together form a 3 to 8 membered ring which optionally comprises in addition to the nitrogen atom one or more further heteroatoms, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 to 3 double bonds, and/or is optionally interrupted by one or more —C(O)— groups,
Q is a phenyl, naphthyl or a monocyclic or bicyclic heteroaryl ring,
R⁶ is
(i) hydrogen or hydroxy, or
(ii) a C₁-C₆-alkyl, C₃-C₆-alkenyl, C₃-C₆-alkynyl or C₁-C₆-alkoxy radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
R⁷ is hydrogen or a C₁-C₆-alkyl radical, R⁸ and R⁹ are independently of one another
(i) hydrogen and/or
(ii) a C₁-C₆-alkyl radical, C₂-C₆-alkenyl, C₃-C₈-cycloalkyl and/or phenyl ring, a heterocyclyl ring having 3 to 8 ring atoms and/or a monocyclic heteroaryl ring, optionally substituted one or more times, identically or differently, by hydroxy, —NR¹⁰R¹¹, cyano, halogen, —CF₃, C₁-C₆-alkoxy and/or —OCF₃, or
R⁸ and R⁹ together with the nitrogen atom form a 5- to 7-membered ring which optionally comprises in addition to the nitrogen atom 1 or 2 further heteroatoms, and which may be substituted one or more times, identically or differently, by hydroxy, —NR¹⁰R¹¹, cyano, halogen, —CF₃, C₁-C₆-alkoxy and/or —OCF₃,
R¹⁰ and R¹¹ are independently of one another hydrogen or a C₁-C₆-alkyl radical which is optionally substituted one or more times, identically or differently, by hydroxy, cyano, halogen, —CF₃, C₁-C₆-alkoxy and/or —OCF₃,
R¹², R¹³, R¹⁴ are independently of one another a C₁-C₆-alkyl, C₂-C₆-alkenyl and/or C₂-C₆-alkynyl radical, a C₃-C₇-cycloalkyl or phenyl ring, a heterocyclyl ring having 3 to 8 ring atoms or a monocyclic heteroaryl ring, optionally in each case themselves substituted one or more times, identically or differently, by hydroxy, nitro, —NR⁸R⁹, cyano, halogen, —CF₃, C₁-C₆-alkyl, C₁-C₆-alkoxy and/or —OCF₃,
R¹⁶ is
(i) hydrogen or
(ii) a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
and the salts, diasteriomers thereof.

2. Compound of the general formula (1) according to claim 1, in which Z is the group —NH—, represented by the formula (1a)

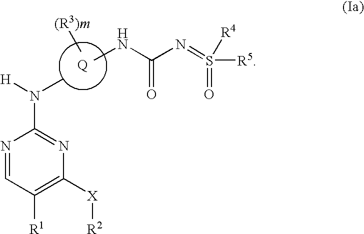

3. Compounds of the general formula (1) according to claim 1, in which Z is a direct bond, represented by the formula (1b)

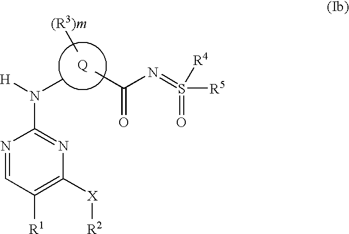

4. Compounds of the general formula (Ia) according to claim 2,
in which
R¹ is halogen, —CF₃, —OCF₃, C₁-C₄-alkyl or nitro,
R² is a C₁-C₁₀-alkyl, C₂-C₁₀-alkenyl or C₂-C₁₀-alkynyl radical, a C₃-C₇-cycloalkyl, phenyl or a mono- or bicyclic heteroaryl ring or a heterocyclyl ring having 3 to 7 ring atoms,
in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy
R³ is
(i) hydroxy, halogen, cyano, nitro, —CF₃, —OCF₃, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl, C₂-C₆-alkenyl, C₂-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, C₁-C₆-alkoxy, and/or C₁-C₆-alkyl, or
R⁴ and R⁵ together with the sulphur form a 3 to 7-membered ring which is optionally substituted one or more times, identically or differently, by hydroxy, C₁-C₆-alkyl, C₁-C₆-alkoxy or —NR⁸R⁹,
X is —O—, —S— or —NR¹⁵—,
where
R¹⁵ is
(i) hydrogen or
(ii) a C₁-C₆-alkyl radical, C₃-C₈-cycloalkyl or phenyl ring, a heterocyclyl ring having 3 to 8 ring atoms or a monocyclic heteroaryl ring, or
(iii) —C(O)—(C₁-C₆)-alkyl, —C(O)-phenyl, or —C(O)-benzyl, and (ii) and (iii) are optionally substituted one or more times, identically or differently, by hydroxy, —NR¹⁰R¹¹, cyano, halogen, —CF₃, C₁-C₆-alkoxy and/or —OCF₃, or
if X is —NR¹⁵—, alternatively —NR¹⁵— and R² together form a 3 to 8 membered ring which optionally comprises in addition to the nitrogen atom one or more further heteroatoms, is optionally substituted one or more times, identically or differently, by hydroxy, C₁-C₆-alkyl, C₁-C₆-alkoxy, —C(O)R¹², —SO₂R¹², halogen or the group —NR⁸R⁹, and/or is optionally interrupted by one or more —C(O)— groups,
Q is a phenyl or a monocyclic or bicyclic heteroaryl ring,
R⁶ is a C₂-C₅-alkyl, C₄-C₆-alkenyl, C₄-C₆-alkynyl or C₂-C₅-alkoxy radical, a C₄-C₆-cycloalkyl or phenyl ring, a heterocyclyl ring having 3 to 5 ring atoms or a monocyclic heteroaryl ring,
in each case optionally themselves substituted one or more times, identically or differently, by hydroxy, —NR⁸R⁹, cyano, halogen, —CF₃, C₁-C₆-alkoxy and/or —OCF₃,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or phenyl ring, and/or a monocyclic heteroaryl ring,
in each case optionally substituted one or more times, identically or differently, by hydroxy, —NR¹⁰R¹¹ or C₁-C₆-alkoxy, or
R⁸ and R⁹ together with the nitrogen atom form a 5- to 7-membered ring which optionally comprises in addition to the nitrogen atom 1 further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy,
R¹² is a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,
in each case optionally themselves substituted one or more times, identically or differently, by hydroxy, halogen, nitro, —NR⁸R⁹, C₁-C₆-alkyl, and/or C₁-C₆-alkoxy,
R¹³ and R¹⁴ are independently of one another a C₁-C₆-alkyl radical, and
R¹⁶ is a C₁-C₆-alkyl radical, a C₃-C₇-cycloalkyl or phenyl ring, a heterocyclyl ring having 3 to 8 ring atoms or a monocyclic heteroaryl ring,
and the salts, diastereomers and enantiomers thereof.

5. Compounds according to claim 1, in which
Q is a phenyl ring,
and the salts, diastereomers and enantiomers thereof.

6. Compounds according to claim 1, in which
R¹ is bromine,
and the salts, diastereomers and enantiomers thereof.

7. Compounds according to claim 1, in which
R² is a C₁-C₆-alkyl radical, by hydroxy,
and the salts, diastereomers and enantiomers thereof.

8. Compounds according to claim 1, in which
X is —O— or —NR¹⁵—, where R¹⁵ is hydrogen,
and the salts, diastereomers and enantiomers thereof.

9. Compounds according to claim 1, in which
R³ is halogen or is a C₁-C₃-alkyl and/or C₁-C₃-alkoxy radical,
and the salts, diastereomers and enantiomers thereof.

10. Compounds according to claim 1, in which
m is 0 or 1,
and the salts, diastereomers and enantiomers thereof.

11. Compounds according to claim 1, in which
R⁴ and R⁵ are independently of one another a C₁-C₅-alkyl, C₂-C₅-alkenyl, C₂-C₅-alkynyl radical, a C₃-C₆-cycloalkyl or phenyl ring, a heterocyclyl ring having 3 to 6 ring atoms or a monocyclic heteroaryl ring, or
R⁴ and R⁵ together with the sulphur form a 3 to 7-membered ring,
and the salts, diastereomers and enantiomers thereof.

12. Compounds according to claim 1, in which
R⁴ and R⁵ are independently of one another a C₁-C₆-alkyl radical,
and the salts, diastereomers and enantiomers thereof.

13. Compounds according to claim 1, in which
R⁷ is hydrogen or a C₁-C₆-alkyl radical,
and the salts, diastereomers and enantiomers thereof.

14. Compounds according to claim 1, in which
R⁸ and R⁹ are hydrogen and/or a C₁-C₆-alkyl radical, a C₃-C₆-cycloalkyl and/or phenyl ring, and/or a monocyclic heteroaryl ring, or
R⁸ and R⁹ form together with the nitrogen atom a 5- or 6-membered ring which optionally comprises in addition to the nitrogen atom 1 further heteroatom,
and the salts, diastereomers and enantiomers thereof.

15. Compounds according to claim 1, in which
R¹² is a C₁-C₆-alkyl radical, a phenyl or monocyclic heteroaryl ring, in each case optionally themselves substituted one or more times, identically or differently, by hydroxy, halogen, nitro or C₁-C₆-alkyl, and the salts, diastereomers and enantiomers thereof.

16. Compounds according to any of claim 1, in which
R¹⁶ is a C₁-C₆-alkyl radical,
and the salts, diastereomers and enantiomers thereof.

17. Compounds of the general formula (I) according to claim 1 in which
R¹ is halogen, —CF₃ or a monocyclic heteroaryl ring which is optionally substituted one or more times, identically or differently, by hydroxy, —NR⁸R⁹, —NR⁷—C(O)—R², —NR⁷—C(O)—OR¹², —NR⁷—C(O)—NR⁸R⁹, —NR⁷—SO₂—R², cyano, halogen, —CF₃, C₁-C₆-alkoxy, —OCF₃ and/or C₁-C₆-alkyl,
R² is a C₁-C₁₀-alkyl radical or bicyclic heteroaryl ring, in each case optionally substituted one or more times, identically or differently, by hydroxy, —NR⁸R⁹, —NR⁷—C(O)—R¹² and/or a C₁-C₄-alkyl radical which is optionally itself substituted one or more times by hydroxy
R³ is halogen and/or a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹,
m is 0 or 1,
R⁴ and R⁵ are independently of one another a C₁-C₆-alkyl radical, in each case optionally itself substituted one or more times, identically or differently, by hydroxy, —NR⁸R⁹, C₁-C₆-alkoxy, and/or C₁-C₆-alkyl,
X is —O— or —NH—,
Q is a phenyl ring,
Z is the group —NH— or a direct linkage,
R⁷ is hydrogen or a C₁-C₆-alkyl radical,
R⁸ and R⁹ are each independently of one another hydrogen and/or a C₁-C₄-alkyl radical, C₃-C₆-cycloalkyl and/or a phenyl ring, and/or a monocyclic heteroaryl ring, in each case optionally substituted one or more times, identically or differently, by hydroxy, —NR¹⁰R¹¹ or C₁-C₆-alkoxy, or
R⁸ and R⁹ together with the nitrogen atom form a 5- to 7-membered ring which optionally comprises in addition to the nitrogen atom one further heteroatom, and which may be substituted one or more times by hydroxy,
R¹⁰ and R¹¹ are independently of one another hydrogen or a C₁-C₆-alkyl radical which is optionally substituted one or more times, identically or differently, by hydroxy,
R¹² is a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy,
and the salts, diastereomers and enantiomers thereof.

18. Compounds according to formula (I) of claim 1, in which
R¹ is halogen,
R² is a C₁-C₁₀-alkyl radical, substituted identically or differently by hydroxy or a C₁-C₆-alkyl radical,
R³ is halogen, or is a C₁-C₃-alkyl and/or C₁-C₆-alkoxy radical,
m is 0 or 1,
R⁴ and R⁵ are independently of one another a C₁-C₆-alkyl radical,
X is —O— or —NR¹⁵—, where R¹⁵ is hydrogen,
Q is a phenyl ring,
and the salts, diastereomers and enantiomers thereof.

19. Process for preparing compounds according to claim 1 by reacting 2-chloropyrimidines of the formula (II) with nucleophiles of the formula (III) to give compounds of the formula (I) 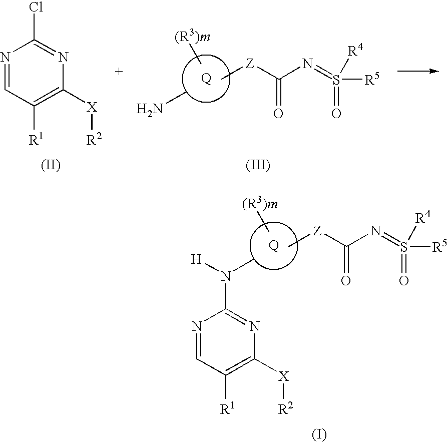
where Q, R¹, R², Z, R³, R⁴, R⁵, X and m have the meanings indicated in the general formula (I) according to claim 1.

20. Intermediates of the formula (II): 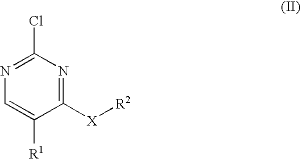
where R¹, R² and X have the meanings indicated in the general formula (I) according to claim 1.

21. Process for preparing intermediates of the formula (II) by reacting 2,4-dichloropyrimidines of the formula (V) with nucleophiles of the formula (IV) 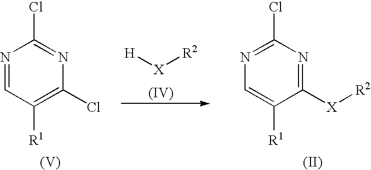
where R¹, R² and X have the meanings indicated in the general formula (I) according to claim 1.

22. Intermediates of the formula (IIIa): 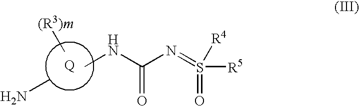
where Q, R³, R⁴ and R⁵ have the meanings indicated in the general formula (I) according to claim 1.

23. Intermediates of the formula (IIIb): 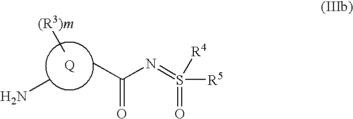
where Q, R³, R⁴ and R⁵ have the meanings indicated in the general formula (I) according to claim 1.

24. Process for preparing intermediates of the formula (IIIa) comprising the steps
a) reaction of an isocyanate of the formula (VII) with a sulphoximine of the formula (VIII) to give an intermediate of the formula (VI) 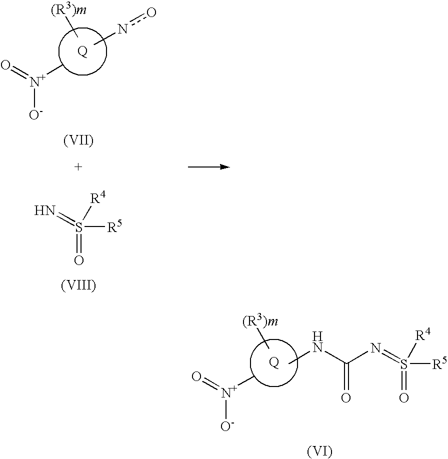
b) reduction of the nitro group to obtain the intermediates of the formula (IIIa) 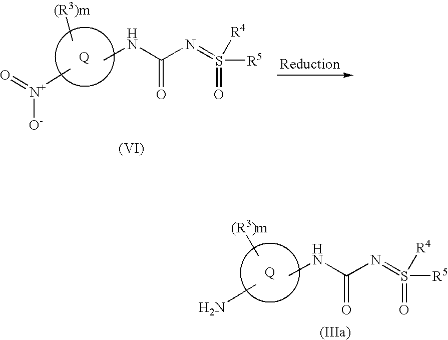
where Q, R³, R⁴ and R⁵ have the meanings indicated in the general formula (I) according the claim 1.

25. Process foe preparing intermediates of the formula (IIIb) comprising the steps
a) reaction of an acid chloride of the formula (IX) with sulphoximines of the formula (VIII) to give intermediates of the formula (X) 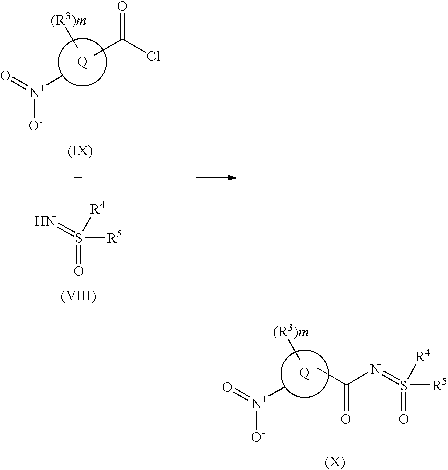
b) reduction of the nitro group to obtain the intermediates of the formula (IIIb) 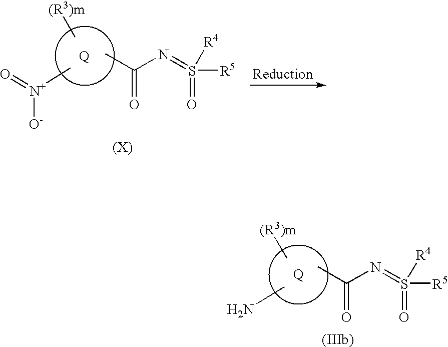
where Q, R³, R⁴ and R⁵ have the meanings indicated in the general formula (I) according to claim 1.

26. Compounds according to claim 1 for use as medicaments.

27. Use of a compound according to claim 1 for producing a medicament for the treatment of cancer.

28. Pharmaceutical formulation comprising a compound according to claim 1.

29. A method of treating cancer comprising administering a compound according to claim 1.
